(19) United States
(12) Patent Application Publication
(10) Pub. No.: US 2026/0317307 A1
(43) Pub. Date: Oct. 15, 2026

(54) Process for the Preparation of Deucravacitinib, and Crystalline Forms Thereof

(71) Applicant: Alivus Life Sciences Limited, Mohol Bazarpeth, Solapur (IN)

(72) Inventors: Venkata Raghavendra Charyulu Palle, Maharashtra (IN); Pratik R. Patel, Vadodara (IN); Srinivas Harinath Harathi Dwaraka Nath, Telangana (IN); Shivaji Haribhau Shelke, Maharashtra (IN); Sachin Mahadeo Lad, Maharashtra (IN); Surendra Sitaram Mundekar, Maharashtra (IN); Amit A. Kudale, Maharashtra (IN); Shridhar Maruti Shingan, Maharashtra (IN); Kiran Rajendra Ahire, Maharashtra (IN)

(21) Appl. No.: 19/485,071

(22) PCT Filed: May 15, 2024
(86) PCT No.: PCT/IB2024/054712

§ 371 Date: Nov. 17, 2025

(30) Foreign Application Priority Data
May 15, 2023 (IN) 202321033892
Jun. 12, 2023 (IN) 202321040060
Nov. 9, 2023 (IN) 202321076754

(51) Int. Cl. C07D 403/12

(52) U.S. Cl. CPC C07D 403/12; C07B 2200/05; C07B 2200/13

(57) ABSTRACT

The present invention relates to a process for the preparation of deucravacitinib. The present invention also relates to a novel intermediate of deucravacitinib, process for its preparation, and use thereof in the process for the preparation of deucravacitinib. The present invention further relates to novel crystalline forms of deucravacitinib, and processes for their preparation. Further, the present invention relates to a process for the preparation of crystalline form A of deucravacitinib.

### PRIORITY

This application claims the benefit of Indian Provisional Applications 202321033892 filed on May 15, 2023, entitled “PROCESS FOR THE PREPARATION OF DEUCRAVACITINIB AND INTERMEDIATES THEREOF”, 202321040060 filed on Jun. 12, 2023, entitled “PROCESS FOR THE PREPARATION OF DEUCRAVACITINIB AND INTERMEDIATES THEREOF”, and 202321076754 filed on Nov. 9, 2023, entitled “CRYSTALLINE FORMS OF DEUCRAVACITIB AND PROCESSES FOR THEIR PREPARATION”, the contents of which are incorporated herein by reference.

### BACKGROUND OF THE INVENTION

### Technical Field

The present invention relates to a process for the preparation of deucravacitinib. The present invention also relates to a novel intermediate of deucravacitinib, process for its preparation, and use thereof for the preparation of deucravacitinib. The present invention further relates to novel crystalline forms of deucravacitinib, and processes for their preparation. Also, the present invention relates to pharmaceutical compositions containing therapeutically effective amount of the novel crystalline forms of deucravacitinib and at least one pharmaceutically acceptable excipient. Further, the present invention relates to process for the preparation of crystalline form A of deucravacitinib.

### Description of the Related Art

Deucravacitinib, also known by its chemical name N-[4-[6-(cyclopropanecarbonylamido)-4-[2-methoxy-3-(1-methyl-1,2,4-triazol-3-yl)anilino]-N-(trideuteriomethyl) pyridazine-3-carboxamide, is represented by the following structure of formula I.

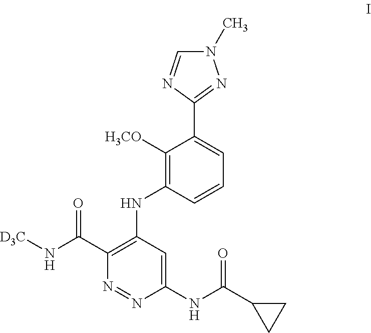 [SMILES: [2H]C([2H])([2H])NC(=O)c1nnc(NC(=O)C2CC2)cc1Nc1cccc(-c2ncn(C)n2)c1OC]

Deucravacitinib is a tyrosine kinase 2 (TYK2) inhibitor indicated for the treatment of adults with moderate-to-severe plaque psoriasis who are candidates for systemic therapy or phototherapy. Deucravacitinib is also being evaluated in Phase II/Phase III clinical trials in patients with psoriatic arthritis, ulcerative colitis, Crohn's disease, Sjogren's syndrome, lupus erythematosus, hidradenitis suppurativa, and alopecia areata. This indicates deucravacitinib to be a promising therapeutic agent.

Deucravacitinib is disclosed in International Publication No. WO 2014/074661A1 (the WO '661 publication). The synthesis of deucravacitinib is described in the WO '661 publication. Alternate process for the preparation of deucravacitinib is described in International Publication No. WO 2018/183649A1 (the WO '649 publication).

Solid state forms of deucravacitinib are known in the art. International Publication No. WO 2018/183656A1 (the WO '656 publication) relates to crystalline form A of deucravacitinib, WO 2022/165141A1 (the WO '141 publication) relates to crystalline form E of deucravacitinib, WO 2022/212181A1 (the WO '181 publication) relates to various crystalline forms of deucravacitinib, and processes for the preparation thereof.

In light of deucravacitinib being a promising therapeutic agent, and the known fact that different solid forms of active pharmaceutical substances provide numerous advantages, such as improved solubility, dissolution profile, better stability, shelf-life etc., there exists a need to provide industrially applicable improved processes for the preparation of deucravacitinib, and also, novel crystalline forms of deucravacitinib having beneficial properties.

Accordingly, the object of the present invention is to provide a novel process which is a convenient and efficient method for the preparation of deucravacitinib, via a novel intermediate compound as described herein. Also, the object of the present invention is to provide novel crystalline forms of deucravacitinib.

### SUMMARY OF THE INVENTION

The present invention provides a process for the preparation of deucravacitinib, a compound of formula I (the “compound I”) or a pharmaceutically acceptable salt thereof,

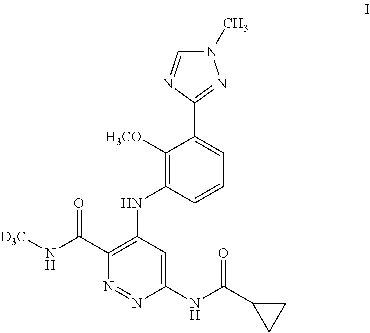 [SMILES: [2H]C([2H])([2H])NC(=O)c1nnc(NC(=O)C2CC2)cc1Nc1cccc(-c2ncn(C)n2)c1OC]

comprising reacting a compound of formula II (the “compound II”),

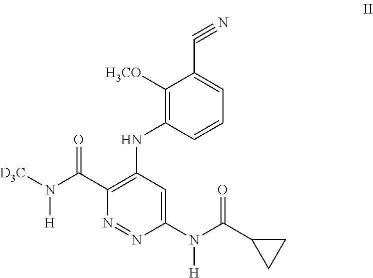 [SMILES: [2H]C([2H])([2H])NC(=O)c1nnc(NC(=O)C2CC2)cc1Nc1cccc(C#N)c1OC]

with a N-methylhydrazine compound, which may be substituted or unsubstituted, to obtain deucravacitinib, the compound I, and optionally converting it to a pharmaceutically acceptable salt thereof.

The present invention also provides a process for the preparation of a compound of formula II (the “compound II”),

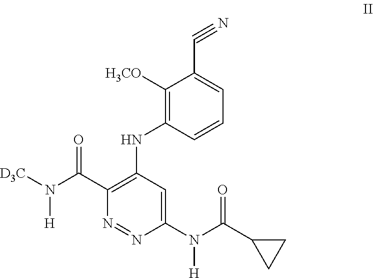 [SMILES: [2H]C([2H])([2H])NC(=O)c1nnc(NC(=O)C2CC2)cc1Nc1cccc(C#N)c1OC]

the process comprising the steps of:
(a) reacting a compound of formula IV (the “compound IV”),

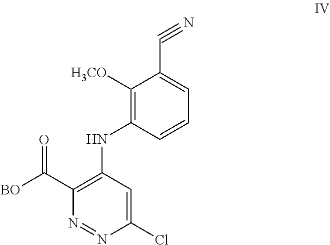

wherein B is selected from the group consisting of H, C₁₋₆ alkyl, Li, Na, K, Mg, Ca and Zn; with deuterated methylamine of formula CD₃NH₂ or a salt thereof to obtain a compound of formula III (the “compound III”); and

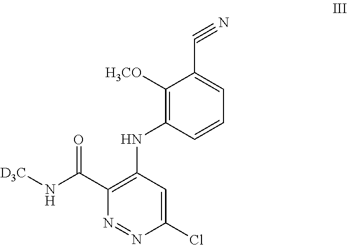 [SMILES: [2H]C([2H])([2H])NC(=O)c1nnc(Cl)cc1Nc1cccc(C#N)c1OC]

(b) reacting the compound III with 1-cyclopropanecarboxamide in the presence of a reagent selected from a precatalyst, or a catalyst and a ligand, to obtain the compound II.

The present invention further provides a process for the preparation of a compound of formula IV (the “compound IV”),

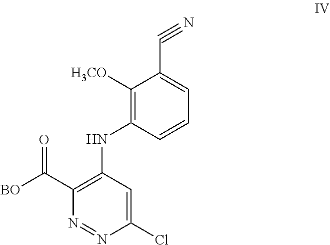

wherein B is selected from the group consisting of H, C₁₋₆ alkyl, Li, Na, K, Mg, Ca and Zn; comprising reacting a compound of formula VI (the “compound VI”) with a compound of formula V (the “compound V”) or a hydrate thereof,

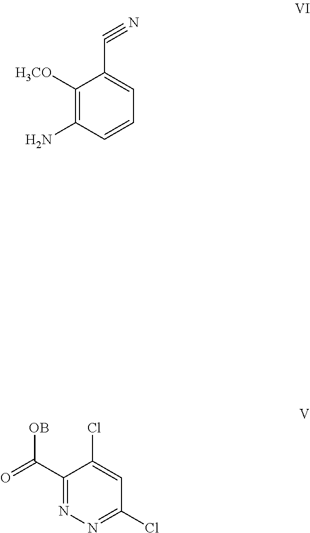

wherein B is as defined above; to obtain the compound IV.

The present invention also provides a compound of formula IV,

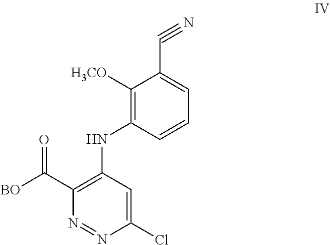

wherein B is selected from the group consisting of H, C₁₋₆ alkyl, Li, Na, K, Mg, Ca and Zn.

The present invention also provides use of a compound of formula IV (the “compound IV”),

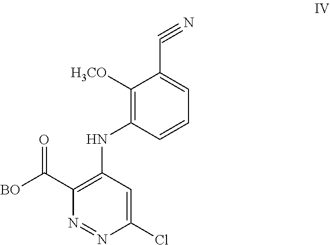

wherein B is selected from the group consisting of H, C₁₋₆ alkyl, Li, Na, K, Mg, Ca and Zn; in the preparation of deucravacitinib (the compound I) or a pharmaceutically acceptable salt thereof.

The present invention further provides a process for the preparation of deucravacitinib, a compound of formula I (the “compound I”), as described above, or a pharmaceutically acceptable salt thereof, comprising converting a compound of formula IV (the “compound IV”),

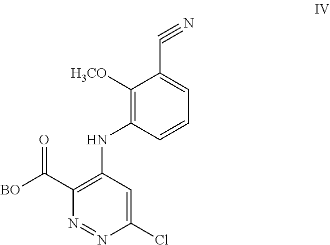

wherein B is selected from the group consisting of H, C₁₋₆ alkyl, Li, Na, K, Mg, Ca and Zn; to the compound I, the process comprising the steps of:
(a-1) reacting the compound IV with deuterated methylamine of formula CD₃NH₂ or a salt thereof to obtain a compound of formula III (the “compound III”);

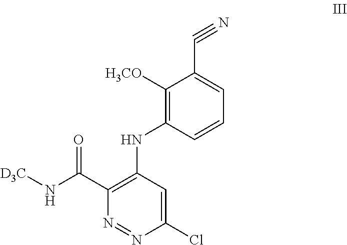 [SMILES: [2H]C([2H])([2H])NC(=O)c1nnc(Cl)cc1Nc1cccc(C#N)c1OC]

(b-1) reacting the compound III obtained in step (a-i) with 1-cyclopropanecarboxamide in the presence of a reagent selected from a precatalyst, or a catalyst and a ligand, to obtain a compound of formula II (the “compound II”); and

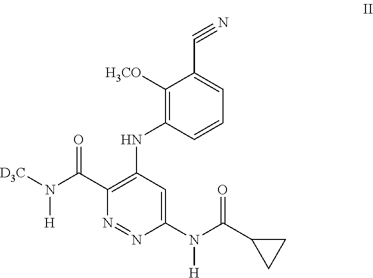 [SMILES: [2H]C([2H])([2H])NC(=O)c1nnc(NC(=O)C2CC2)cc1Nc1cccc(C#N)c1OC]

(c-1) reacting the compound II obtained in step (b-1) with a N-methylhydrazine compound, which may be substituted or unsubstituted, to obtain deucravacitinib, the compound I, and optionally converting it to a pharmaceutically acceptable salt thereof.

The present invention provides use of a compound of formula II (the “compound II”),

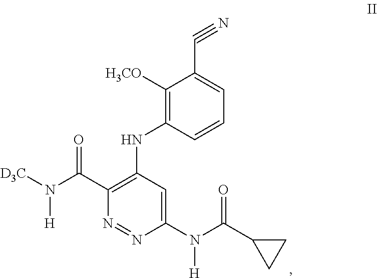 [SMILES: [2H]C([2H])([2H])NC(=O)c1nnc(NC(=O)C2CC2)cc1Nc1cccc(C#N)c1OC]

in the preparation of deucravacitinib (the compound I) or a pharmaceutically acceptable salt thereof.

The present invention also provides use of a compound of formula VI (the “compound VI”) in the preparation of deucravacitinib or a salt thereof, wherein the compound VI is prepared by reacting a compound of formula VII (the “compound VII”) with phosphorous oxychloride,

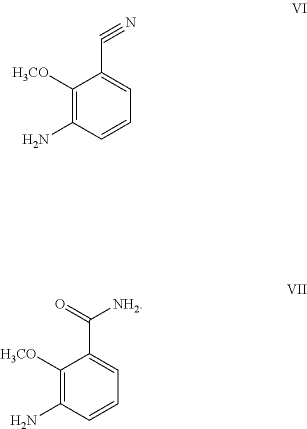 [SMILES: COc1c(N)cccc1C#N ; COc1c(N)cccc1C(N)=O]

The present invention further provides a process for the preparation of deucravacitinib, a compound of formula I (the “compound I”), as described above, or a pharmaceutically acceptable salt thereof, the process comprising the steps of:
(1) reacting a compound of formula III (the “compound III”),

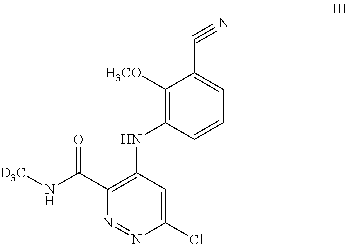 [SMILES: [2H]C([2H])([2H])NC(=O)c1nnc(Cl)cc1Nc1cccc(C#N)c1OC]

with a N-methylhydrazine compound, which may be substituted or unsubstituted, to obtain a compound of formula XI (the “compound XI”); and

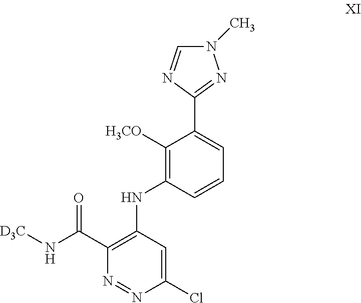 [SMILES: [2H]C([2H])([2H])NC(=O)c1nnc(Cl)cc1Nc1cccc(-c2ncn(C)n2)c1OC]

(2) reacting the compound XI with 1-cyclopropanecarboxamide in the presence of a reagent selected from a precatalyst, or a catalyst and a ligand, to obtain deucravacitinib, the compound I, and optionally converting it to a pharmaceutically acceptable salt thereof.

The present invention also provides a process for the preparation of a compound of formula III (the “compound III”),

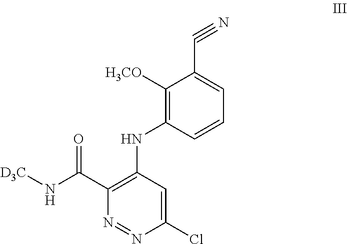 [SMILES: [2H]C([2H])([2H])NC(=O)c1nnc(Cl)cc1Nc1cccc(C#N)c1OC]

comprising reacting a compound of formula IV (the “compound IV”),

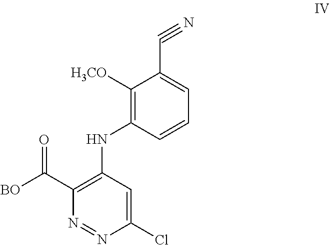

wherein B is selected from the group consisting of H, C₁₋₆ alkyl, Li, Na, K, Mg, Ca and Zn; with deuterated methylamine of formula CD₃NH₂ or a salt thereof in the presence of a coupling agent to obtain the compound III.

The present invention further provides a process for the preparation of deucravacitinib, a compound of formula I (the “compound I”), as described above, or a pharmaceutically acceptable salt thereof, the process comprising the steps of:
(a-2) reacting the compound IV, as described above, with deuterated methylamine of formula CD₃NH₂ or a salt thereof to obtain a compound of formula III (the “compound III”), as described above;
(b-2) reacting the compound III obtained in the step (a-2) with a N-methylhydrazine compound, which may be substituted or unsubstituted, to obtain a compound of formula XI (the “compound XI”), as described above; and
(c-2) reacting the compound XI obtained in the step (b-2) with 1-cyclopropanecarboxamide in the presence of a reagent selected from a precatalyst, or a catalyst and a ligand, to obtain deucravacitinib, the compound I, and optionally converting it to a pharmaceutically acceptable salt thereof.

The present invention also provides a process for the preparation of a compound of formula XI (the “compound XI”), as described above, or an acid addition salt thereof, comprising reacting a compound of formula III (the “compound III”) with a N-methylhydrazine compound, which may be substituted or unsubstituted, to obtain the compound XI, which is optionally reacted with an acid to obtain the acid addition salt thereof.

The present invention also provides use of a compound of formula XI (the “compound XI”) in the preparation of deucravacitinib (the compound I), as described above, or a pharmaceutically acceptable salt thereof, wherein the compound XI is prepared by reacting the compound III, as described above, with a N-methylhydrazine compound, which may be substituted or unsubstituted, to obtain the compound XI; which is converted to deucravacitinib, the compound I.

The present invention further provides a process for the preparation of a compound of formula XII (the “compound XII”),

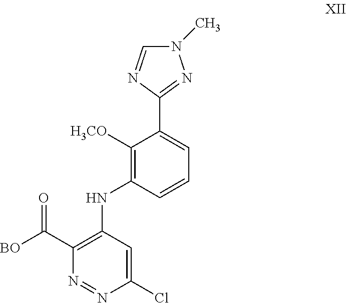

wherein B is selected from the group consisting of H, C₁₋₆ alkyl, Li, Na, K, Mg, Ca and Zn; comprising reacting the compound IV, as

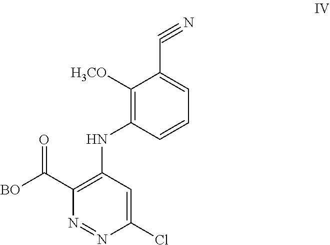

wherein B is as defined above; with a N-methylhydrazine compound, which may be substituted or unsubstituted, to obtain the compound XII.

The present invention further provides a process for the preparation of deucravacitinib, a compound of formula I (the “compound I”), as described above, or a pharmaceutically acceptable salt thereof, the process comprising the steps of:
(A-1) reacting a compound of formula VII (the “compound VII”) with phosphorous oxychloride to obtain a compound of formula VI (the “compound VI”);

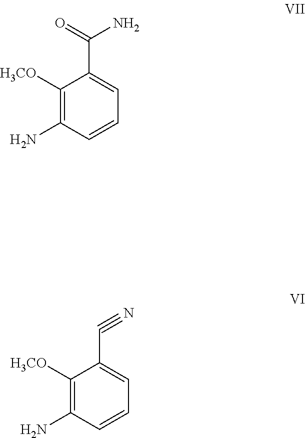 [SMILES: COc1c(N)cccc1C(N)=O ; COc1c(N)cccc1C#N]

(B-1) reacting the compound VI obtained in the step (A-1) with a compound of formula V (the “compound V”) or a hydrate thereof, to obtain a compound of formula IV (the “compound IV”);

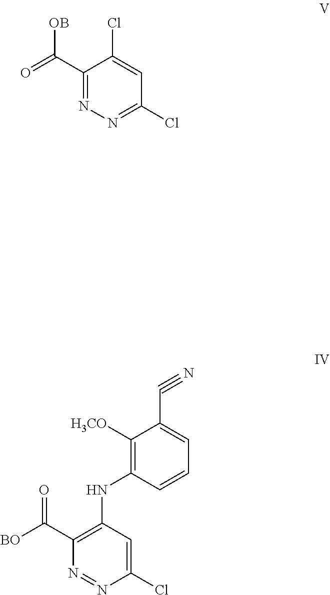

wherein B is selected from the group consisting of H, C₁₋₆ alkyl, Li, Na, K, Mg, Ca and Zn; (C-1) reacting the compound IV obtained in the step (B-1) with deuterated methylamine of formula CD₃NH₂ or a salt thereof to obtain a compound of formula III (the “compound III”);

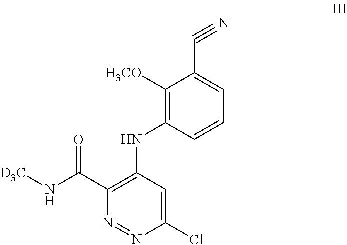 [SMILES: [2H]C([2H])([2H])NC(=O)c1nnc(Cl)cc1Nc1cccc(C#N)c1OC]

(D-1a) reacting the compound III obtained in the step (C-1) with a N-methylhydrazine compound which may be substituted or unsubstituted, to obtain a compound of formula XI (the “compound XI”); and

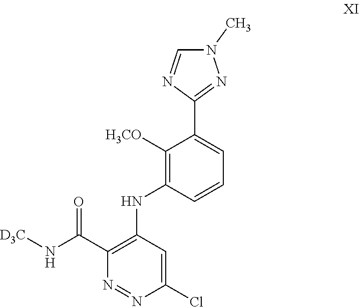 [SMILES: [2H]C([2H])([2H])NC(=O)c1nnc(Cl)cc1Nc1cccc(-c2ncn(C)n2)c1OC]

(E-1a) reacting the compound XI obtained in the step (D-1a) with 1-cyclopropanecarboxamide in the presence of a reagent selected from a precatalyst, or a catalyst and a ligand, to obtain deucravacitinib, the compound I, and optionally converting it to a pharmaceutically acceptable salt thereof.

The present invention still further provides a process for the preparation of deucravacitinib, a compound of formula I (the “compound I”), as described above, or a pharmaceutically acceptable salt thereof, the process comprising the steps of:
(A-1) reacting a compound of formula VII (the “compound VII”) with phosphorous oxychloride to obtain a compound of formula VI (the “compound VI”);

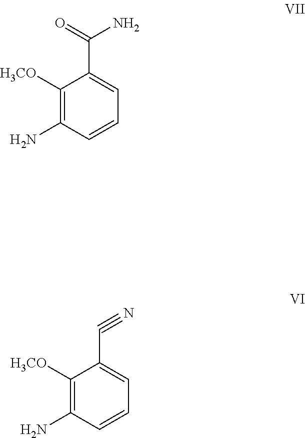 [SMILES: COc1c(N)cccc1C(N)=O ; COc1c(N)cccc1C#N]

(B-1) reacting the compound VI obtained in the step (A-1) with a compound of formula V (the “compound V”) or a hydrate thereof, to obtain a compound of formula IV (the “compound IV”);

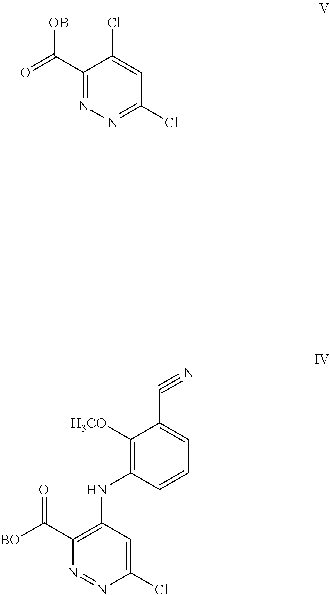

wherein B is selected from the group consisting of H, C₁₋₆ alkyl, Li, Na, K, Mg, Ca and Zn; (C-1) reacting the compound IV obtained in the step (B-1) with deuterated methylamine of formula CD₃NH₂ or a salt thereof to obtain a compound of formula III (the “compound III”);

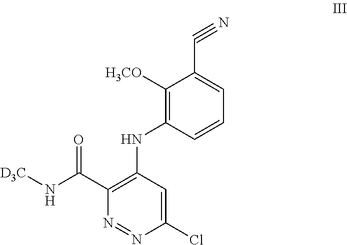 [SMILES: [2H]C([2H])([2H])NC(=O)c1nnc(Cl)cc1Nc1cccc(C#N)c1OC]

(D-1b) reacting the compound III obtained in the step (C-1) with 1-cyclopropanecarboxamide in the presence of a reagent selected from a precatalyst, or a catalyst and a ligand, to obtain a compound of formula II (the “compound II”); and

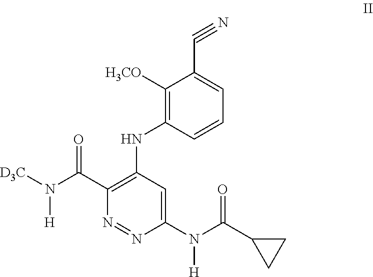 [SMILES: [2H]C([2H])([2H])NC(=O)c1nnc(NC(=O)C2CC2)cc1Nc1cccc(C#N)c1OC]

(E-1b) reacting the compound II obtained in the step (D-1b) with a N-methylhydrazine compound which may be substituted or unsubstituted, to obtain deucravacitinib, the compound I, and optionally converting it to a pharmaceutically acceptable salt thereof.

The present invention also provides a process for the preparation of deucravacitinib, a compound of formula I (the “compound I”), as described above, the process comprising the steps of:
(a-3) reacting a compound of formula V (the “compound V”), wherein B is selected from the group consisting of H, C₁₋₆ alkyl, Li, Na, K, Mg, Ca and Zn; with deuterated methylamine of formula CD₃NH₂ or a salt thereof in the presence of a coupling agent at a temperature of about −10° C. to about 0° C. to obtain a compound of formula XIII (the “compound XIII”);

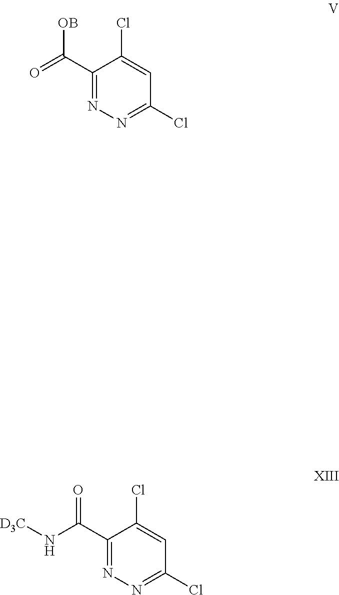

(b-3) reacting the compound XIII with a compound of formula XIV (the “compound XIV”) in the presence of a base to obtain a compound of formula XI (the “compound XI”); and

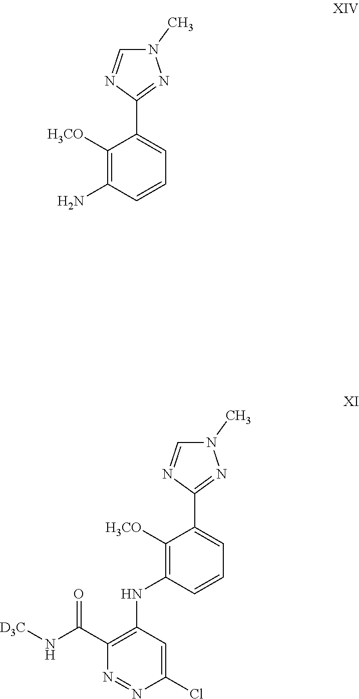 [SMILES: COc1c(N)cccc1-c1ncn(C)n1 ; [2H]C([2H])([2H])NC(=O)c1nnc(Cl)cc1Nc1cccc(-c2ncn(C)n2)c1OC]

(c-3) reacting the compound XI with 1-cyclopropanecarboxamide in the presence of a reagent and a base; wherein the reagent is selected from a precatalyst, or a catalyst and a ligand; and the base is selected from sodium carbonate or potassium carbonate, to obtain deucravacitinib, the compound I, in a purity of ≥95%, and wherein the content of impurity A represented by the following chemical structure,

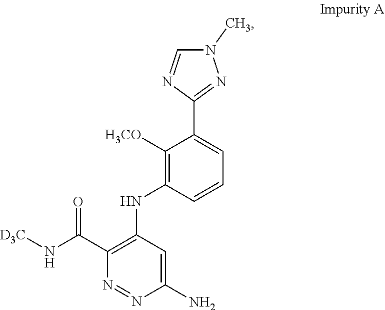 [SMILES: [2H]C([2H])([2H])NC(=O)c1nnc(N)cc1Nc1cccc(-c2ncn(C)n2)c1OC]

is less than 0.15% w/w as determined by High Performance Liquid Chromatography (HPLC).

The present invention further provides a process for the preparation of crystalline form A of deucravacitinib, wherein the process comprises:
(A) dissolving deucravacitinib in a first solvent selected from acetic acid or dimethyl sulfoxide, optionally in the presence of an additional solvent, at a temperature of about 40° C. to about 70° C. to form a solution;
(B) combining the solution of step (A) with a second solvent selected from a lower alcohol, a nitrile solvent, water, or a mixture thereof, at a temperature of about 40° C. to about 70° C. to obtain a mixture;
(C) cooling the mixture of step (B) to a temperature from about 30° C. to about 5° C.;
(D) obtaining crystalline form A of deucravacitinib from the mixture of step (C); and
(E) isolating the crystalline form A of deucravacitinib as obtained in the step (D), wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta.

The present invention provides a novel crystalline form of deucravacitinib designated as Form GL1 (hereinafter referred to as “crystalline form GL1” or “form GL1”), which is characterized by an X-ray powder diffraction (XRPD) pattern having peaks at 3.34, 7.71, 12.1, 23.4, and 25.6±0.2 degrees 2 theta.

The present invention also provides another novel crystalline form of deucravacitinib, designated as form GL2 (hereinafter referred to as “crystalline form GL2” or “form GL2”), which is characterized by an X-ray powder diffraction (XRPD) pattern having peaks at 8.5, 11.7, 18.0, 23.4, and 24.3±0.2 degrees 2 theta.

The present invention provides a process for the preparation of crystalline form A of deucravacitinib, wherein the process comprises:
(A-i) dissolving deucravacitinib in a first solvent selected from acetic acid or dimethyl sulfoxide, optionally in the presence of an additional solvent, at a temperature of about 20° C. to about 35° C. to form a solution;
(B-i) combining the solution of step (A-i) with a second solvent selected from a lower alcohol or water, at a temperature of about 20° C. to about 35° C. to obtain a mixture;
(C-i) obtaining crystalline form A of deucravacitinib from the mixture of step (B-i); and
(D-i) isolating the crystalline form A of deucravacitinib as obtained in the step (C-i),
wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta; and
wherein when the first solvent is acetic acid, then the second solvent is not water.

The present invention also provides a process for the preparation of crystalline form A of deucravacitinib, wherein the process comprises:
(A-ii) providing a solution of a crystalline form of deucravacitinib in a first solvent selected from acetic acid or dimethyl sulfoxide, optionally in the presence of an additional solvent, at a temperature of about 40° C. to about 70° C.; wherein the crystalline form of deucravacitinib is selected from crystalline form GL-1 or crystalline form GL-2;
(B-ii) combining the solution of step (A-ii) with a second solvent selected from a lower alcohol, a nitrile solvent, water, or a mixture thereof, at a temperature of about 40° C. to about 70° C. to obtain a mixture;
(C-ii) cooling the mixture of step (B-ii) to a temperature from about 30° C. to about 5° C.;
(D-ii) obtaining crystalline form A of deucravacitinib from the mixture of step (C-ii); and
(E-ii) isolating the crystalline form A of deucravacitinib as obtained in the step (D-ii),
wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta.

### BRIEF DESCRIPTION OF THE DRAWINGS

FIG. 1 is a characteristic X-ray Powder Diffraction Pattern (XRPD) of crystalline form GL1 of deucravacitinib as obtained in Example 25.

FIG. 2 is a characteristic X-ray Powder Diffraction Pattern (XRPD) of crystalline form GL2 of deucravacitinib as obtained in Example 27.

FIG. 3 is a characteristic X-ray Powder Diffraction Pattern (XRPD) of crystalline form A of deucravacitinib as obtained in Example 28.

### DETAILED DESCRIPTION OF THE INVENTION

In one aspect, the present invention provides a process for the preparation of deucravacitinib, a compound of formula I (the “compound I”), as described above, or a pharmaceutically acceptable salt thereof,

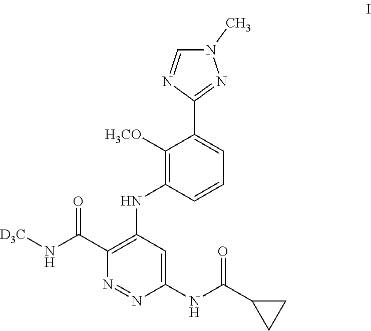 [SMILES: [2H]C([2H])([2H])NC(=O)c1nnc(NC(=O)C2CC2)cc1Nc1cccc(-c2ncn(C)n2)c1OC]

comprising reacting a compound of formula II (the “compound II”), as described above, with a N-methylhydrazine compound, which may be substituted or unsubstituted,
to obtain deucravacitinib, the compound I, and optionally converting it to a pharmaceutically acceptable salt thereof.

In the context of the present invention, the term “room temperature” means a temperature of about 25° C. to about 30° C.

As used herein, the term “about” refers to any value which lies within the range defined by a number up to 10% of the value.

In one embodiment, the substituted N-methylhydrazine compound is prepared by a process comprising reacting N-methylhydrazine with esters of formic acid.

In one embodiment, the esters of formic acid include, but are not limited to, methyl formate, ethyl formate, and the like.

In one embodiment, the substituted N-methylhydrazine compound is N-methyl-N-formylhydrazine.

In one embodiment, the compound II is reacted with the unsubstituted N-methylhydrazine compound in the presence of a formylating agent to obtain deucravacitinib, the compound I.

In one embodiment, the compound II is reacted with the unsubstituted N-methylhydrazine compound to give an intermediate amidine compound which is further contacted with a formylating agent to give deucravacitinib, the compound I.

In one embodiment, the formylating agent includes but is not limited to formic acid, esters of formic acid, acetic formic anhydride, ammonium formate, paraformaldehyde, formaldehyde, and the like.

In one embodiment, the compound II is reacted with the N-methylhydrazine compound, which may be substituted or unsubstituted, in the presence of a base.

In one embodiment, the base is selected from an inorganic base or an organic base.

In one embodiment, the inorganic base is selected from the group consisting of potassium tert-butoxide, sodium tert-butoxide, lithium tert butoxide, lithium carbonate, sodium carbonate, potassium carbonate, sodium bicarbonate, potassium bicarbonate, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), N,N,N′,N′-tetramethyl-1,8-naphthalenediamine, and a mixture thereof.

In one embodiment, the base is inorganic base selected from potassium tert-butoxide, sodium tert-butoxide or lithium tert butoxide.

In one embodiment, the base is potassium tert-butoxide.

In one embodiment, a mixture of the compound II and the N-methylhydrazine compound is added to the base.

In one embodiment, the base is added to a mixture of the compound II and the N-methylhydrazine compound.

In one embodiment, the reaction of the compound II with the N-methylhydrazine compound is carried out in the presence of a solvent.

In one embodiment, the solvent includes but is not limited to ether solvents such as diethyl ether, diisopropyl ether, methyl tert-butyl ether, tetrahydrofuran, dioxane and the like; ester solvents such as methyl acetate, ethyl acetate, n-propyl acetate, isopropyl acetate, tert-butyl acetate and the like; hydrocarbon solvents such as toluene, xylene, chlorobenzene, heptane, hexane and the like; ketone solvents such as acetone, ethyl methyl ketone, methyl isobutyl ketone and the like; alcohol solvents such as methanol, ethanol, n-propyl alcohol, isopropyl alcohol, n-butyl alcohol, isobutyl alcohol, sec-butyl alcohol, tert-butyl alcohol, pentanol, octanol and the like; haloalkane solvents such as dichloromethane, chloroform, ethylene dichloride, and the like; nitrile solvents such as acetonitrile, propionitrile, butyronitrile, and the like; dimethyl sulfoxide; dimethylacetamide; N-methyl-2-pyrrolidone; water; or mixtures thereof.

In one embodiment, the solvent used is an ether solvent, an ester solvent, a ketone solvent or a hydrocarbon solvent.

In one embodiment, the solvent used is an ether solvent.

In one embodiment, the reaction of the compound II with the N-methylhydrazine compound is carried out at a temperature of about 10° C. to about 60° C.

In one embodiment, the reaction of the compound II with the N-methylhydrazine compound is carried out for a period that range from about 3 hours to about 16 hours, or longer.

In one embodiment, the compound II used in the process for the preparation of deucravacitinib (the compound I), is prepared by a process comprising the steps of:
(a) reacting a compound of formula IV (the “compound IV”), as described above, with deuterated methylamine of formula CD₃NH₂ or a salt thereof to obtain a compound of formula III (the “compound III”), as described above; and
(b) reacting the compound III with 1-cyclopropanecarboxamide in the presence of a reagent selected from a precatalyst, or a catalyst and a ligand, to obtain the compound II.

In one embodiment, the salt of deuterated methylamine is deuterated methylamine hydrochloride salt.

In one embodiment, the step (a) is carried out in the presence of a coupling agent.

In one embodiment, the coupling agent used in the step (a) is selected from the group consisting of a carbodiimide reagent, an anhydride reagent, a benzotriazole reagent, a phosphorus reagent, a borane reagent, a quinolone reagent and a mixture thereof.

In one embodiment, the coupling agent is carbodiimide reagent, which includes, but is not limited to EDC (N-(3-dimethylaminopropyl)-N′-ethylcarbodiimide), DCC (dicyclohexylcarbodiimide), DIC (diisopropylcarbodiimide) and the like.

In one embodiment, the coupling agent is anhydride reagent, such as T₃P (propylphosphonic anhydride).

In one embodiment, the coupling agent is benzotriazole reagent, which includes, but is not limited to HBTU (N,N,N′,N′-tetramethyl-O-(1H-benzotriazol-1-yl)uronium hexafluorophosphate), HATU (1-[Bis(dimethylamino)methylene]-1H-1,2,3-triazolo[4,5-b]pyridinium 3-oxide hexafluorophosphate), HOBt (hydroxybenzotriazole), TBTU (O-(benzotriazol-1-yl)-N,N,N′,N′-tetramethyluronium tetrafluoroborate), TATU (O-(7-azabenzotriazole-1-yl)-1,1,3,3-tetramethyluronium tetrafluoroborate), PyBOP ((benzotriazol-1-yloxy) tripyrrolidinophosphonium hexafluorophosphate), TDBTU (O-(3,4-dihydro-4-oxo-1,2,3-benzotriazin-3-yl)-N,N,N′,N′-tetramethyluronium tetrafluoroborate), HDMC (N-[(5-Chloro-3-oxido-1H-benzotriazol-1-yl)-4-morpholinylmethylene]-N-methyl-methanaminium hexafluorophosphate), HCTU (2-(6-chloro-1H-benzotriazole-1-yl)-1,1,3,3-tetramethylaminium hexafluorophosphate), DEPBT (3-(diethoxyphosphoryloxy)-1,2,3-benzotriazin-4(3H)-one), PyAOP ((7-azabenzotriazol-1-yloxy)tripyrrolidinophosphonium hexafluorophosphate), BOP (benzotriazol-1-yloxytris(dimethylamino)phosphonium hexafluorophosphate), HOOBt (hydroxy-3,4-dihydro-4-oxo-1,2,3-benzotriazine), HOSu (N-hydroxysuccinimide), HOAt (1-hydroxy-7-azabenzotriazole) and the like.

In one embodiment, the coupling agent is phosphorus reagent, which includes, but is not limited to COMU ((1-cyano-2-ethoxy-2-oxoethylidenaminooxy)dimethylamino-morpholino-carbenium hexafluorophosphate), HOTT (S-(1-oxido-2-pyridyl)-N,N,N′,N′-tetramethylthiuronium hexafluorophosphate), PyCIU (chlorodipyrrolidinocarbenium hexafluorophosphate), TFFH (tetramethylfluoroformamidinium hexafluorophosphate), FDPP (pentafluorophenyl diphenylphosphinate) and the like.

In one embodiment, the coupling agent is borate reagent, which includes, but is not limited to DMTMM (4-(4,6-dimethoxy-1,3,5-triazin-2-yl)-4-methylmorpholinium tetrafluoroborate), TSTU (N,N,N,N-tetramethyl-O—(N-succinimidyl)uronium tetrafluoroborate), TPTU (O-(2-oxo-1 (2H)pyridyl)-N,N,N′,N′-tetramethyluronium tetrafluoroborate), TOTU (O-[(ethoxycarbonyl)cyanomethylenamino]-N,N,N′,N′-tetramethyluronium tetrafluoroborate) and the like.

In one embodiment, the coupling agent is quinoline reagent, which includes, but is not limited to IIDQ (isobutyl 1,2-dihydro-2-isobutoxy-1-quinolinecarboxylate), EEDQ (N-Ethoxycarbonyl-2-ethoxy-1,2-dihydroquinoline) and the like.

In one embodiment, the coupling agent used in the step (a) is selected from a carbodiimide reagent or a benzotriazole reagent.

In one embodiment, the coupling agent used in the step (a) is EDC, HOBt, or a mixture thereof.

In one embodiment, the step (a) may be optionally carried out in the presence of a base.

In one embodiment, the base used in the step (a) is selected from an inorganic base or an organic base. The inorganic base and the organic base are as discussed above.

In one embodiment, the step (a) is carried out at a temperature of about 10° C. to about 60° C.

In one embodiment, the step (a) is carried out for a period that may range from about 3 hours to about 16 hours, or longer.

In one embodiment, in the step (a), the compound IV wherein B is Zn, is reacted with deuterated methylamine of formula CD₃NH₂.

In one embodiment, in the step (b), the compound III is reacted with 1-cyclopropanecarboxamide in the presence of a reagent selected from a precatalyst, or a catalyst and a ligand, to obtain the compound II.

In one embodiment, the catalyst used in the step (b) includes, but is not limited to, palladium catalyst, copper catalyst, nickel catalyst, cobalt catalyst, and the like.

In one embodiment, the catalyst is the palladium catalyst which is selected from the group consisting of Pd(OAc)₂, PdCh(MeCN)₂, Pd₂(dba)₃, Pd(dba)₂, [(Allyl)PdCl]₂, [(Crotyl)PdCl]₂, and a mixture thereof.

In one embodiment, in the step (b), the catalyst used is Pd(OAc)₂.

In one embodiment, in the step (b), the ligand used is phosphine ligand, which includes, but is not limited to, Josiphos, SEGPHOS ((2H,2′H-[4,4′-Bi-1,3-benzodioxole]-5,5′-diyl)bis(diphenylphosphane)), SDP ((R/S)-(−)-7,7′-Bis(diphenylphosphino)-2,2′,3,3′-tetrahydro-1,1′-spirobiindene, DPEphos (Bis[(2-diphenylphosphino) phenyl]ether), Taniaphos [((R_{P})-1-[(R)-α-(Dimethylamino)-2-(diphenylphosphino)benzyl]-2-diphenylphosphinoferrocene) OR ((S_{P})-1-[(S)-α-(Dimethylamino)-2-(diphenylphosphino)benzyl]-2-diphenylphosphinoferrocene)], Xantphos (4,5-Bis(diphenylphosphino)-9,9-dimethylxanthene), DPPF (1,1′-Ferrocenediyl-bis(diphenylphosphine)), DCyPF ([1,1′-Bis(di-cyclohexylphosphino)ferrocene]), BINAP (2,2′-bis(diphenylphosphino)-1,1′-binaphthyl).

In one embodiment, Josiphos ligand includes, but is not limited to, SL-J009-1 [(R)-1-[(S)-2-(Dicyclohexylphosphino)ferrocenyl]ethyldi-tert-butylphosphine]; SL-J002 [(S)-1-[(R)-2-(Diphenylphosphino)ferrocenyl]ethyldi-tert-butylphosphine]; BRETTPhos [2-(Dicyclohexylphosphino)3,6-dimethoxy-2′,4′,6′-triisopropyl-1,1′-biphenyl]; DCEPhos [Bis(dicyclohexylphosphinophenyl) ether]; and the like.

In one embodiment, the catalyst can be incorporated in the ligand forming a precatalyst, that can be used in the reaction of the compound III with 1-cyclopropanecarboxamide to obtain the compound II.

In one embodiment, the precatalyst used in the step (b) is selected from Josiphos SL-J009-1 Pd G3 [{(R)-1-[(Sp)-2(dicyclohexylphosphino)ferrocenyl]ethylditert-butylphosphine}[2-(2′-amino-1,1′biphenyl)]palladium(ii) Methanesulfonate], XPhos Pd G3 [(2-Dicyclohexylphosphino-2′,4′,6′-triisopropyl-1,1′-biphenyl)[2-(2′-amino-1,1′-biphenyl)]palladium(II) methanesulfonate], and XPhos Pd G2 [Chloro(2-dicyclohexylphosphino-2′,4′,6′-triisopropyl-1,1′-biphenyl)[2-(2′-amino-1,1′-biphenyl)]palladium(II), X-Phos aminobiphenyl palladium chloride].

In one embodiment, the step (b) is carried out in the presence of a base.

In one embodiment, the base is selected from an inorganic base or an organic base or a mixture thereof.

In one embodiment, the inorganic base is selected from the group consisting of potassium tert-butoxide, sodium tert-butoxide, lithium tert butoxide, lithium carbonate, sodium carbonate, potassium carbonate, sodium bicarbonate, potassium bicarbonate, cesium carbonate, tripotassium phosphate, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), and N,N,N′,N′-tetramethyl-1,8-naphthalenediamine, TMG (1,1,3,3-tetramethylguanidine), and a mixture thereof.

In one embodiment, the base used is sodium carbonate, potassium carbonate, DABCO (1,4-diazabicyclo[2.2.2]octane), DBU (1,8-diazabicyclo[5.4.0]undec-7-ene), or a mixture thereof.

In one embodiment, the base used is potassium carbonate, DBU, or a mixture thereof.

In one embodiment, the step (b) is carried out at a temperature of about 20° C. to about 100° C.

In one embodiment, the step (b) is carried out for a period ranging from about 3 hours to about 16 hours, or longer.

In one aspect, the compound IV used in the step (a) is prepared by reacting a compound of formula VI (the “compound VI”) with a compound of formula V (the “compound V”) or a hydrate thereof, to obtain the compound IV;

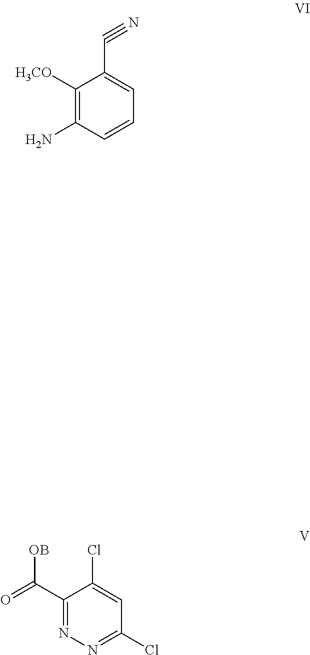

wherein B is selected from the group consisting of H, C₁₋₆ alkyl, Li, Na, K, Mg, Ca and Zn.

As used herein, the term “hydrate” means x·H₂O, wherein x ranges from 0.5 to 6.

In one embodiment, the compound VI is reacted with the compound V in the presence of a base, a metal salt of an acid, or a mixture thereof.

In one embodiment, the base is selected from an inorganic base or an organic base.

In one embodiment, the inorganic base is selected from the group consisting of potassium tert-butoxide, sodium tert-butoxide, lithium tert butoxide, lithium carbonate, sodium carbonate, potassium carbonate, sodium bicarbonate, potassium bicarbonate, cesium carbonate, tripotassium phosphate, sodium methoxide, potassium methoxide, sodium ethoxide, potassium ethoxide, LiHMDS (lithium bis(trimethylsilyl)amide), KHMDS (potassium bis(trimethylsilyl)amide), NaHMDS (sodium bis(trimethylsilyl)amide), lithium hexamethyldisilyazide, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), N,N,N′,N′-tetramethyl-1,8-naphthalenediamine, TMG (1,1,3,3-tetramethylguanidine), and a mixture thereof.

In one embodiment, the metal salt of acid is selected from the group consisting of zinc acetate, chromium acetate, cobalt acetate, and nickel acetate.

In one embodiment, the compound VI is reacted with the compound V, wherein B is H, Li, Na, K, Mg, Ca and Zn, in the presence of zinc acetate to obtain the compound IV, wherein B is Zn.

In one embodiment, the compound VI is reacted with the compound V, wherein B is Li, in the presence of zinc acetate to obtain the compound IV, wherein B is Zn.

In one embodiment, the compound VI is reacted with the compound V, wherein B is H, in the presence of zinc acetate to obtain the compound IV wherein B is Zn.

In one embodiment, the compound VI is reacted with the compound V, wherein B is Na, in the presence of zinc acetate to obtain the compound IV wherein B is Zn.

In one embodiment, the reaction of the compound VI with the compound V is carried out in the presence of a solvent.

In one embodiment, the solvent is selected from the group consisting of an alcohol such as methanol, ethanol, 1-propanol, 2-propanol, n-butanol, t-butanol and the like; an ether such as tetrahydrofuran, dioxane and the like; water; and a mixture thereof.

In one embodiment, the solvent is selected from 2-propanol, water, or a mixture thereof.

In one embodiment, the reaction of the compound VI with the compound V is carried out at a temperature of about 20° C. to about 100° C.

In one embodiment, the reaction of the compound VI with the compound V is carried out at a temperature of about 50° C. to about 75° C.

In one embodiment, the reaction may be stirred for a suitable time. The stirring time may range from about 5 hours to about 40 hours, or longer.

In one aspect, the compound VI is prepared by reacting a compound of formula VII (the “compound VII”), as described above, with phosphorous oxychloride to obtain the compound VI.

In another aspect, the present invention provides a process for the preparation of a compound of formula II (the “compound II”), as described above, the process comprising the steps of:
(a) reacting a compound of formula IV (the “compound IV”), as described above, with deuterated methylamine of formula CD₃NH₂ or a salt thereof to obtain a compound of formula III (the “compound III”), as described above; and
(b) reacting the compound III with 1-cyclopropanecarboxamide in the presence of a reagent selected from a precatalyst, or a catalyst and a ligand, to obtain the compound II.

The steps (a) and (b) are carried out as discussed herein above.

In another aspect, the present invention provides a process for the preparation of a compound of formula IV (the “compound IV”), as described above, comprising reacting a compound of formula VI (the “compound VI”), as described above, with a compound of formula V (the “compound V”), as described above, or a hydrate thereof; to obtain the compound IV.

The reaction of the compound VI with the compound V is carried out as discussed herein above.

In another aspect, the present invention provides a process for the preparation of a compound of formula VI (the “compound VI”), as described above, comprising reacting a compound of formula VII (the “compound VII”), as described above, with phosphorous oxychloride to obtain the compound VI.

In one embodiment, the reaction of the compound VII with phosphorus oxychloride is carried out at a temperature of about 60° C. to about 100° C.

In one embodiment, the reaction is carried out at a temperature of about 65° C. to about 85° C.

In one embodiment, the reaction of the compound VII with phosphorus oxychloride may be carried out for a period of about 6 hours to about 15 hours.

In one embodiment, the reaction is carried out over a period ranging from about 10 hours to about 12 hours.

In one embodiment, the reaction of the compound VII with phosphorus oxychloride is carried out in the absence of a solvent.

In one embodiment, the compound VII is prepared by a process comprising the steps of:
(i) reacting a compound of formula X (the “compound X”) with a nitrating agent to obtain a compound of formula IX (the “compound IX”);

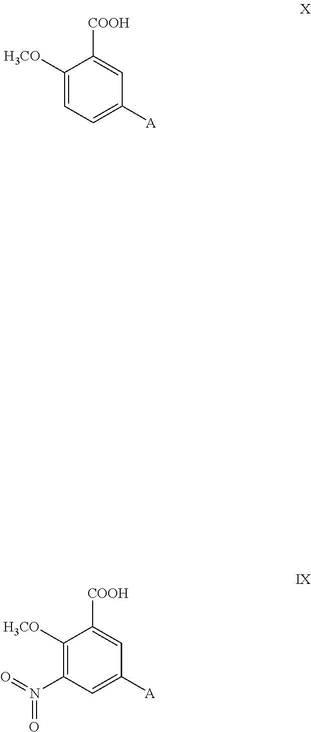

wherein A is selected from the group consisting of H, Cl, Br and I.
(ii) converting the compound IX obtained in the step (i) to a compound of formula VIII (the “compound VIII”) in the presence of a coupling agent and a source of ammonia; and

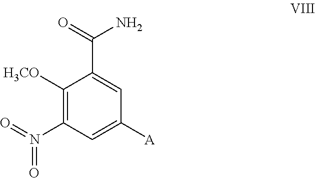

(iii) subjecting the compound VIII obtained in the step (ii) to a hydrogenation reaction to obtain the compound VII.

In one embodiment, in the step (i), the nitrating agent is a mixture of nitric acid with an agent selected from sulfuric acid, phosphorous pentoxide, or acetic anhydride.

In one embodiment, in the step (ii), the coupling agent is selected from the group consisting of thionyl chloride, oxalyl chloride, phosphorus pentachloride, phosphorus trichloride, phosphorus oxychloride, sulfuryl chloride, oxalyl bromide, phosphorus oxybromide, phosphorus tribromide and a mixture thereof.

In one embodiment, the conversion of the compound IX to the compound VIII is carried out by treating the compound IX with the coupling agent to obtain an activated carboxylic acid thereof, which on treatment with a source of ammonia gives the compound VIII.

In one embodiment, the term “activated carboxylic acid” refers to an intermediate compound with a labile leaving group.

In one embodiment, the activated carboxylic acid of the compound IX is an acid halide.

In one embodiment, the source of ammonia is selected from the group consisting of formamide, aqueous ammonia, methanolic ammonia, and a mixture thereof.

In one embodiment, in the step (iii), the hydrogenation reaction is carried out in the presence of a catalyst selected from the group consisting of palladium, platinum, Raney nickel, and a mixture thereof.

In one aspect, the present invention provides a compound of formula IV (the “compound IV”),

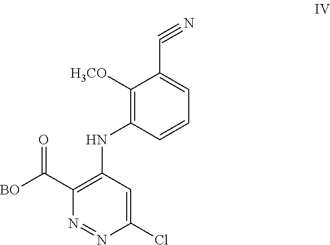

wherein B is selected from the group consisting of H, C₁₋₆ alkyl, Li, Na, K, Mg, Ca and Zn.

In one embodiment, the compound IV is a hydrate.

In one embodiment, the present invention provides a compound of formula IV, wherein B is Zn.

In one embodiment, the present invention provides the compound IV wherein B is Zn, characterized by a proton NMR (CDCl₃) spectrum having peaks at δ: 12.13 (br s, 1H), 7.927 (d, 1H), 7.68 (d, 1H), 7.36 (t, 1H), 7.31 (s, 1H), 3.91 (s, 3H).

In one aspect, the present invention provides use of a compound of formula IV (the “compound IV”),

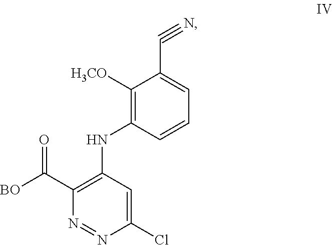

wherein B is selected from the group consisting of H, C₁₋₆ alkyl, Li, Na, K, Mg, Ca and Zn; in the preparation of deucravacitinib (the compound I) or a pharmaceutically acceptable salt thereof.

In another aspect, the present invention provides a process for the preparation of deucravacitinib, a compound of formula I (the “compound I”), as described above, or a pharmaceutically acceptable salt thereof, comprising converting a compound of formula IV (the “compound IV”),

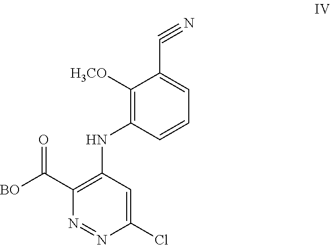

wherein B is selected from the group consisting of H, C₁₋₆ alkyl, Li, Na, K, Mg, Ca and Zn; to the compound I, the process comprising the steps of:
(a-1) reacting the compound IV with deuterated methylamine or a salt thereof to obtain a compound of formula III (the “compound III”);

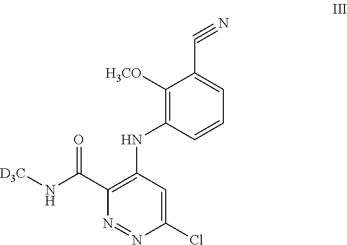 [SMILES: [2H]C([2H])([2H])NC(=O)c1nnc(Cl)cc1Nc1cccc(C#N)c1OC]

(b-1) reacting the compound III obtained in the step (a-1) with 1-cyclopropanecarboxamide in the presence of a reagent selected from a precatalyst, or a catalyst and a ligand, to obtain a compound of formula II (the “compound II”); and

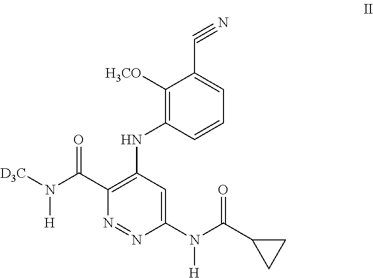 [SMILES: [2H]C([2H])([2H])NC(=O)c1nnc(NC(=O)C2CC2)cc1Nc1cccc(C#N)c1OC]

(c-1) reacting the compound II obtained in the step (b-1) with a N-methylhydrazine compound, which may be substituted or unsubstituted, to obtain deucravacitinib, the compound I, and optionally converting it to a pharmaceutically acceptable salt thereof.

The steps (a-1), (b-1) and (c-1) are carried out as discussed herein above.

In one aspect, the present invention provides use of a compound of formula II (the “compound II”),

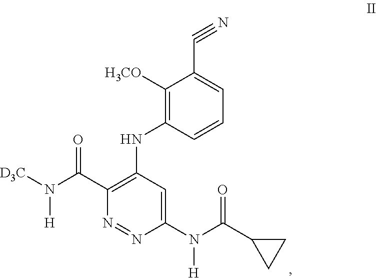 [SMILES: [2H]C([2H])([2H])NC(=O)c1nnc(NC(=O)C2CC2)cc1Nc1cccc(C#N)c1OC]

in the preparation of deucravacitinib (the compound I) or a pharmaceutically acceptable salt thereof.

In another aspect, the present invention provides a process for the preparation of deucravacitinib or a pharmaceutically acceptable salt thereof, comprising converting a compound of formula II (the “compound II”), as described above, to deucravacitinib, the compound I.

In one embodiment, the conversion of the compound II to deucravacitinib, the compound I can be carried out by following any of the following methods:
(1a) reacting the compound II with N-methyl-N-fonnylhydrazine to obtain deucravacitinib, the compound I; or
(2a) reacting the compound II with N-methylhydrazine and a formylating agent to obtain deucravacitinib, the compound I; or
(3a) converting the nitrile function of the compound II into the amidine function and contacting the obtained compound with dimethylformamide in the presence of a catalyst to obtain deucravacitinib, the compound I; or
(4a) converting the nitrile function of the compound II into the amide function and contacting the obtained compound with dimethyl formamide dimethyl acetal and N-methylhydrazine to obtain deucravacitinib, the compound I.

In one aspect, the present invention provides use of the compound VI in the preparation of deucravacitinib or a pharmaceutically acceptable salt thereof, wherein the compound VI is prepared by reacting a compound of formula VII (the “compound VII”) with phosphorous oxychloride,

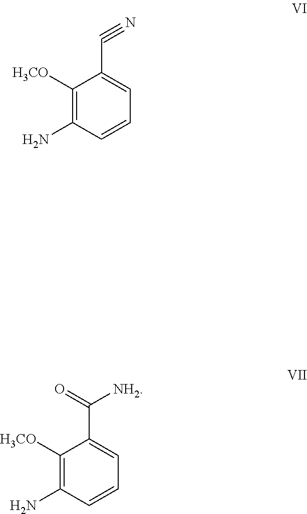 [SMILES: COc1c(N)cccc1C#N ; COc1c(N)cccc1C(N)=O]

In another aspect, the present invention provides a process for the preparation of deucravacitinib (the compound I) or a pharmaceutically acceptable salt thereof, comprising converting a compound of formula VI (the “compound VI”) to deucravacitinib, wherein the compound VI is prepared by reacting a compound of formula VII (the “compound VII”) with phosphorous oxychloride.

In one aspect, the present invention provides a process for the preparation of deucravacitinib, a compound of formula I (the “compound I”), as described above, or a pharmaceutically acceptable salt thereof, the process comprising the steps of:
(1) reacting a compound of formula III (the “compound III”),

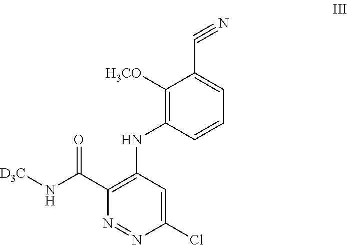 [SMILES: [2H]C([2H])([2H])NC(=O)c1nnc(Cl)cc1Nc1cccc(C#N)c1OC]

with a N-methylhydrazine compound, which may be substituted or unsubstituted, to obtain a compound of formula XI (the “compound XI”), and

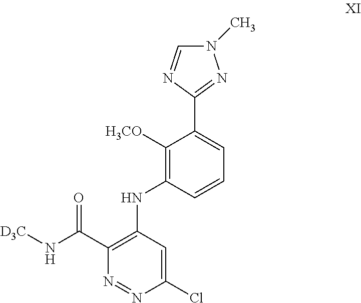 [SMILES: [2H]C([2H])([2H])NC(=O)c1nnc(Cl)cc1Nc1cccc(-c2ncn(C)n2)c1OC]

(2) reacting the compound XI with 1-cyclopropanecarboxamide in the presence of a reagent selected from a precatalyst, or a catalyst and a ligand, to obtain deucravacitinib, the compound I, and optionally converting it to a pharmaceutically acceptable salt thereof.

In one embodiment, in the step (1), the substituted N-methylhydrazine compound is prepared by a process comprising reacting N-methylhydrazine with a formylating agent.

In one embodiment, in the step (1), the substituted N-methylhydrazine compound is N-methyl-N-formylhydrazine.

In one embodiment, in the step (1), the substituted N-methylhydrazine compound is treated with anhydrous sodium sulphate before reaction with the compound III.

In one embodiment, in the step (1), the compound III is reacted with the unsubstituted N-methylhydrazine compound in the presence of a formylating agent to obtain the compound XI.

In one embodiment, in the step (1), the compound III is reacted with the unsubstituted N-methylhydrazine compound to give a corresponding intermediate compound, which is further treated with the formylating agent to give the compound XI.

In one embodiment, the step (1) is carried out in the presence of a base.

In one embodiment, the base is selected from an inorganic base or an organic base.

In one embodiment, the inorganic base is selected from the group consisting of potassium tert-butoxide, sodium tert-butoxide, lithium tert butoxide, lithium carbonate, sodium carbonate, potassium carbonate, sodium bicarbonate, potassium bicarbonate, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP, DABCO, DBU, DBN, N,N,N′,N′-tetramethyl-1,8-naphthalenediamine, and a mixture thereof.

In one embodiment, the base used is potassium tert-butoxide.

In one embodiment, a mixture of the compound III and the N-methylhydrazine compound is added to the base.

In one embodiment, the base is added to a mixture of the compound III and the N-methylhydrazine compound, which may be substituted or unsubstituted.

In one embodiment, the step (1) is carried out in the presence of a solvent.

In one embodiment, the solvent includes but is not limited to ether solvents such as diethyl ether, diisopropyl ether, methyl tert-butyl ether, tetrahydrofuran, dioxane and the like; ester solvents such as methyl acetate, ethyl acetate, n-propyl acetate, isopropyl acetate, tert-butyl acetate and the like; hydrocarbon solvents such as toluene, xylene, chlorobenzene, heptane, hexane and the like; ketone solvents such as acetone, ethyl methyl ketone, methyl isobutyl ketone and the like; alcohol solvents such as methanol, ethanol, n-propyl alcohol, isopropyl alcohol, n-butyl alcohol, isobutyl alcohol, sec-butyl alcohol, tert-butyl alcohol, pentanol, octanol and the like; haloalkane solvents such as dichloromethane, chloroform, ethylene dichloride, and the like; nitrile solvents such as acetonitrile, propionitrile, butyronitrile, and the like; dimethyl sulfoxide; dimethylacetamide; N-methyl-2-pyrrolidone; water; or mixtures thereof.

In one embodiment, the solvent used is an ether solvent selected from diethyl ether, diisopropyl ether, methyl tert-butyl ether, tetrahydrofuran, or dioxane.

In one embodiment, the reaction of the compound III with the N-methylhydrazine compound, which may be substituted or unsubstituted, is carried out at a temperature ranging from about 10° C. to about 60° C.

In one embodiment, the reaction of the compound III with the N-methylhydrazine compound, which may be substituted or unsubstituted, is carried out over a ranging from about 1 hour to about 16 hours, or longer.

In one embodiment, in the step (2), the compound XI is reacted with 1-cyclopropanecarboxamide in the presence of a reagent selected from a precatalyst, or a catalyst and a ligand, to obtain deucravacitinib, the compound I.

In one embodiment, the catalyst used in the step (2) is as described above for the step (b).

In one embodiment, the catalyst used in the step (2) is Pd(OAc)₂.

In one embodiment, in the step (2), the ligand used is phosphine ligand which includes, but is not limited to, Josiphos, SEGPHOS, SDP, Taniaphos, DPEphos, Xantphos, DPPF, DCyPF, and BINAP.

In one embodiment, the catalyst can be incorporated in the ligand forming a precatalyst, that can be used in the reaction of the compound XI with 1-cyclopropanecarboxamide to obtain deucravacitinib, the compound I.

In one embodiment, the precatalyst used in the step (2) is selected from Josiphos SL-J009-1 Pd G3, XPhos Pd G3, or XPhos Pd G2.

In one embodiment, the step (2) is carried out in the presence of a base.

In one embodiment, the base is selected from an inorganic base or an organic base or a mixture thereof.

In one embodiment, the inorganic base is selected from the group consisting of potassium tert-butoxide, sodium tert-butoxide, lithium tert-butoxide, lithium carbonate, sodium carbonate, potassium carbonate, sodium bicarbonate, potassium bicarbonate, cesium carbonate, tripotassium phosphate, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP, DABCO, DBU, DBN, and N,N,N′,N′-tetramethyl-1,8-naphthalenediamine, TMG, and a mixture thereof.

In one embodiment, the base used is potassium carbonate, DBU, or a mixture thereof.

In one embodiment, the step (2) may be carried out at a temperature ranging from about 20° C. to about 100° C.

In one embodiment, the step (2) may be carried out for a period of about 3 hours to about 16 hours, or longer.

In one embodiment, the deucravacitinib obtained by the process as described herein, may be purified by treatment with aqueous N-Acetyl L-Cysteine solution.

In one aspect, the present invention provides a process for the preparation of a compound of formula III (the “compound III”), as described above, comprising reacting a compound of formula IV (the “compound IV”), as described above, with deuterated methylamine of formula CD₃NH₂ or a salt thereof in the presence of a coupling agent to obtain the compound III.

In another aspect, the present invention provides a process for the preparation of deucravacitinib, a compound of formula I (the “compound I”), as described above, or a pharmaceutically acceptable salt thereof, the process comprising the steps of:
(a-2) reacting the compound IV, as described above, with deuterated methylamine of formula CD₃NH₂ or a salt thereof to obtain a compound of formula III (the “compound III”), as described above;
(b-2) reacting the compound III obtained in the step (a-2) with a N-methylhydrazine compound, which may be substituted or unsubstituted, to obtain a compound of formula XI (the “compound XI”), as described above; and
(c-2) reacting the compound XI obtained in the step (b-2) with 1-cyclopropanecarboxamide in the presence of a reagent selected from a precatalyst, or a catalyst and a ligand, to obtain deucravacitinib, the compound I, and optionally converting it to a pharmaceutically acceptable salt thereof.

In another aspect, the present invention provides a process for the preparation of a compound of formula XI (the “compound XI”) or an acid addition salt thereof,

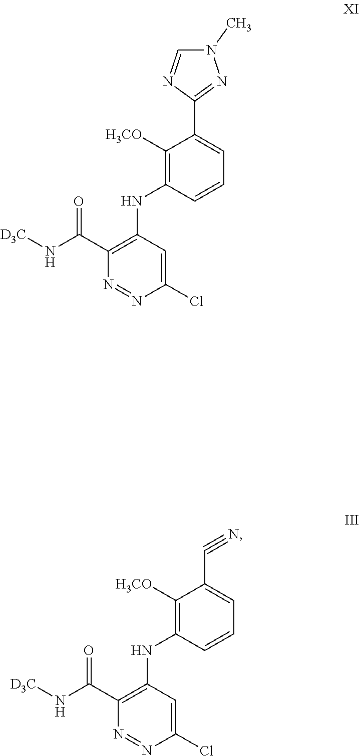 [SMILES: [2H]C([2H])([2H])NC(=O)c1nnc(Cl)cc1Nc1cccc(-c2ncn(C)n2)c1OC ; [2H]C([2H])([2H])NC(=O)c1nnc(Cl)cc1Nc1cccc(C#N)c1OC]

the process comprising reacting a compound of formula III (the “compound III”) with a N-methylhydrazine compound, which may be substituted or unsubstituted, to obtain the compound XI, which is optionally reacted with an acid to obtain the acid addition salt thereof.

In one embodiment, the acid used in the preparation of the acid addition salt of the compound XI, includes but is not limited to sulfuric acid, hydrochloric acid, hydrobromic acid, nitric acid, phosphoric acid, formic acid, acetic acid, propionic acid, methanesulfonic acid, ethanesulfonic acid, ethane-1,2-disulfonic acid, benzenesulfonic acid, p-toluenesulfonic acid, camphor sulfonic acid, naphthalene-2-sulfonic acid, oxalic acid, succinic acid, fumaric acid, maleic acid, tartaric acid, dibenzoyl tartaric acid, lactic acid, mandelic acid, 2-chloromandelic acid, salicylic acid, citric acid, malonic acid, malic acid, adipic acid, gluconic acid, glutaric acid, glutamic acid, palmitic acid and aspartic acid.

In one embodiment, the acid used in the preparation of the acid addition salt of the compound XI is selected from sulfuric acid, hydrochloric acid, hydrobromic acid, nitric acid, phosphoric acid, formic acid, acetic acid, propionic acid, methanesulfonic acid, ethanesulfonic acid, p-toluenesulfonic acid, dibenzoyl tartaric acid.

In another aspect, the present invention provides use of a compound of formula XI (the “compound XI”) in the preparation of deucravacitinib (the compound I) or a pharmaceutically acceptable salt thereof, wherein the compound XI is prepared by reacting a compound of formula III (the “compound III”),

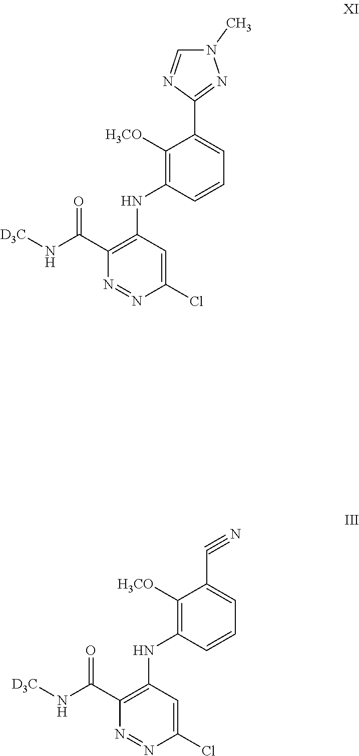 [SMILES: [2H]C([2H])([2H])NC(=O)c1nnc(Cl)cc1Nc1cccc(-c2ncn(C)n2)c1OC ; [2H]C([2H])([2H])NC(=O)c1nnc(Cl)cc1Nc1cccc(C#N)c1OC]

with a N-methylhydrazine compound, which may be substituted or unsubstituted, to obtain the compound XI, which is converted to deucravacitinib, the compound I, and optionally it is converted to a pharmaceutically acceptable salt thereof.

In another aspect, the present invention provides a process for the preparation of deucravacitinib (the “compound I”) or a pharmaceutically acceptable salt thereof, comprising converting a compound of formula XI (the “compound XI”), as described above, to deucravacitinib, wherein the compound XI is prepared by reacting a compound of formula III (the “compound III”), as described above, with a N-methylhydrazine compound, which may be substituted or unsubstituted.

In one aspect, the present invention provides a process for the preparation of a compound of formula XII (the “compound XII”),

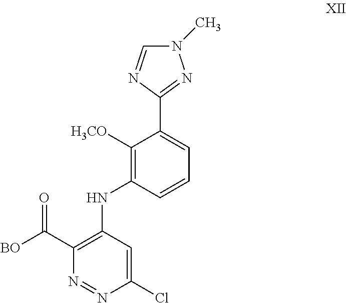

wherein B is selected from the group consisting of H, C₁₋₆ alkyl, Li, Na, K, Mg, Ca and Zn; comprising reacting a compound of formula IV (the “compound IV”),

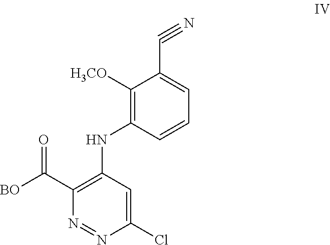

wherein B is as defined above; with a N-methylhydrazine compound, which may be substituted or unsubstituted, to obtain the compound XII.

In one embodiment, the substituted or unsubstituted N-methylhydrazine compound is as described herein.

In one embodiment, the compound IV is reacted with the N-methylhydrazine compound, which may be substituted or unsubstituted, in the presence of a base.

In one embodiment, the base is selected from an inorganic base or an organic base.

The inorganic base and organic base are as described above.

In one embodiment, a mixture of the compound IV and the N-methylhydrazine compound, which may be substituted or unsubstituted, is added to the base.

In one embodiment, the base is added to a mixture of the compound IV and the N-methylhydrazine compound, which may be substituted or unsubstituted.

In one embodiment, the reaction of the compound IV with the N-methylhydrazine compound is carried out at a temperature ranging from about 10° C. to about 60° C.

In one embodiment, the reaction of the compound IV with the N-methylhydrazine compound is carried out over a period ranging from about 3 hours to about 16 hours, or longer.

In another aspect, the present invention provides a process for the preparation of deucravacitinib, a compound of formula I (the “compound I”), as described above, or a pharmaceutically acceptable salt thereof, the process comprising the steps of:
(A-1) reacting a compound of formula VII (the “compound VII”) with phosphorous oxychloride to obtain a compound of formula VI (the “compound VI”);

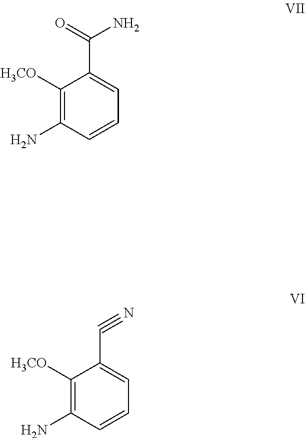 [SMILES: COc1c(N)cccc1C(N)=O ; COc1c(N)cccc1C#N]

(B-1) reacting the compound VI obtained in the step (A-1) with a compound of formula V (the “compound V”) or a hydrate thereof, to obtain a compound of formula IV (the “compound IV”);

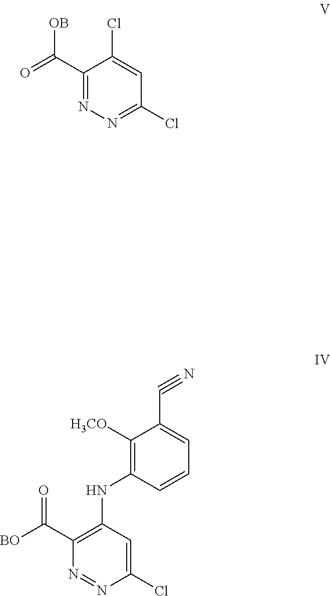

wherein B is selected from the group consisting of H, C₁₋₆ alkyl, Li, Na, K, Mg, Ca and Zn;
(C-1) reacting the compound IV obtained in the step (B-1) with deuterated methylamine of formula CD₃NH₂ or a salt thereof to obtain a compound of formula III (the “compound III”);

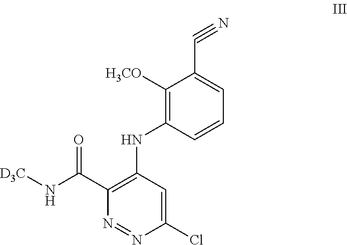 [SMILES: [2H]C([2H])([2H])NC(=O)c1nnc(Cl)cc1Nc1cccc(C#N)c1OC]

(D-1a) reacting the compound III obtained in the step (C-1) with a N-methylhydrazine compound, which may be substituted or unsubstituted, to obtain a compound of formula XI (the “compound XI”); and

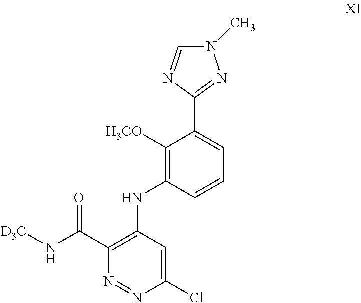 [SMILES: [2H]C([2H])([2H])NC(=O)c1nnc(Cl)cc1Nc1cccc(-c2ncn(C)n2)c1OC]

(E-1a) reacting the compound XI obtained in the step (D-1a) with 1-cyclopropanecarboxamide in the presence of a reagent selected from a precatalyst, or a catalyst and a ligand, to obtain deucravacitinib, the compound I, and optionally converting it to a pharmaceutically acceptable salt thereof.

In another aspect, the present invention provides a process for the preparation of deucravacitinib, a compound of formula I (the “compound I”), as described above, or a pharmaceutically acceptable salt thereof, the process comprising the steps of:
(A-1) reacting a compound of formula VII (the “compound VII”) with phosphorous oxychloride to obtain a compound of formula VI (the “compound VI”);

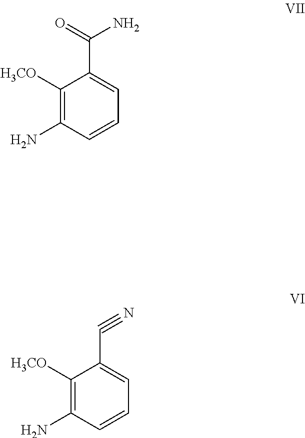 [SMILES: COc1c(N)cccc1C(N)=O ; COc1c(N)cccc1C#N]

(B-1) reacting the compound VI obtained in the step (A-1) with a compound of formula V (the “compound V”) or a hydrate thereof, to obtain a compound of formula IV (the “compound IV”);

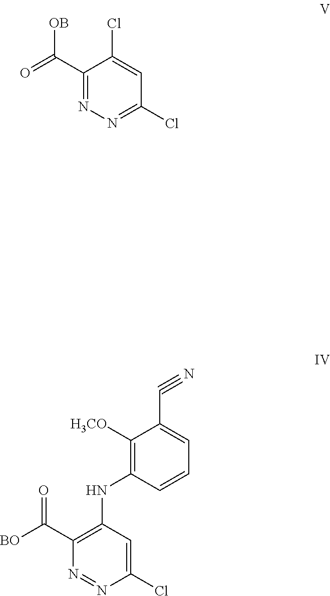

wherein B is selected from the group consisting of H, C₁₋₆ alkyl, Li, Na, K, Mg, Ca and Zn;
(C-1) reacting the compound IV obtained in the step (B-1) with deuterated methylamine of formula CD₃NH₂ or a salt thereof to obtain a compound of formula III (the “compound III”);

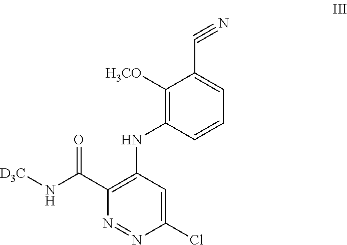 [SMILES: [2H]C([2H])([2H])NC(=O)c1nnc(Cl)cc1Nc1cccc(C#N)c1OC]

(D-1b) reacting the compound III obtained in the step (C-1) with 1-cyclopropanecarboxamide in the presence of a reagent selected from a precatalyst, or a catalyst and a ligand, to obtain a compound of formula II (the “compound II”); and

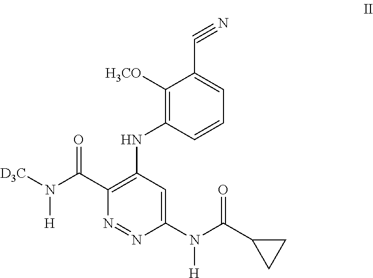 [SMILES: [2H]C([2H])([2H])NC(=O)c1nnc(NC(=O)C2CC2)cc1Nc1cccc(C#N)c1OC]

(E-1b) reacting the compound II obtained in the step (D-1b) with a N-methylhydrazine compound, which may be substituted or unsubstituted, to obtain deucravacitinib, the compound I, and optionally converting it to a pharmaceutically acceptable salt thereof.

In one embodiment, deucravacitinib, the compound I is converted to pharmaceutically acceptable salt of deucravacitinib, comprising reacting deucravacitinib, the compound I with an acid.

In one embodiment, the acid is selected from an organic acid or an inorganic acid.

In one embodiment, the organic acid is selected from oxalic acid, malic acid, maleic acid, malonic acid, tartaric acid, dibenzoyl tartaric acid, fumaric acid, citric acid, malic acid, succinic acid, mandelic acid, lactic acid, formic acid, acetic acid, salicylic acid, propionic acid, 2-chloromandelic acid, p-toluenesulfonic acid, ethane-1,2-disulfonic acid, camphorsulfonic acid, ethanesulfonic acid, methanesulfonic acid, 2-hydroxyethanesulfonic acid, naphthalene-2-sulfonic acid, naphthalene-1,5-disulfonic acid, benzenesulfonic acid, adipic acid, gluconic acid, glutaric acid, glutamic acid, palmitic acid, gentisic acid or aspartic acid; and the inorganic acid is selected from hydrochloric acid, hydrobromic acid, sulfuric acid, nitric acid, or phosphoric acid.

In one embodiment, pharmaceutically acceptable salt of deucravcitinib, may be prepared by any conventional method known in the art.

In one embodiment, the present invention provides a process for the purification of deucravacitinib, the process comprising the steps of:
(1b) reacting deucravacitinib with an organic acid or an inorganic acid to obtain a pharmaceutically acceptable salt of deucravacitinib; and
(2b) reacting the pharmaceutically acceptable salt of deucravacitinib as obtained in the step (1b) with a base to give deucravacitinib.

The organic acid and the inorganic acid used in the step (1b) and the base used in the step (2b) are as discussed above.

In another aspect, the present invention provides a process for the preparation of deucravacitinib, a compound of formula I (the “compound I”), as described above, the process comprising the steps of:
(a-3) reacting a compound of formula V (the “compound V”), wherein B is selected from the group consisting of H, C₁₋₆ alkyl, Li, Na, K, Mg, Ca and Zn; with deuterated methylamine of formula CD₃NH₂ or a salt thereof in the presence of a coupling agent at a temperature of about −10° C. to about 0° C. to obtain a compound of formula XIII (the “compound XIII”);

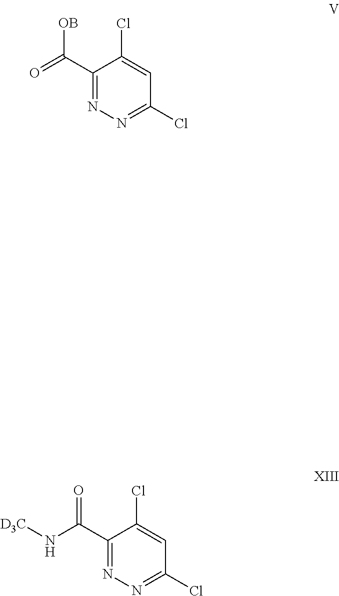

(b-3) reacting the compound XIII with a compound of formula XIV (the “compound XIV”) in the presence of a base to obtain a compound of formula XI (the “compound XI”); and

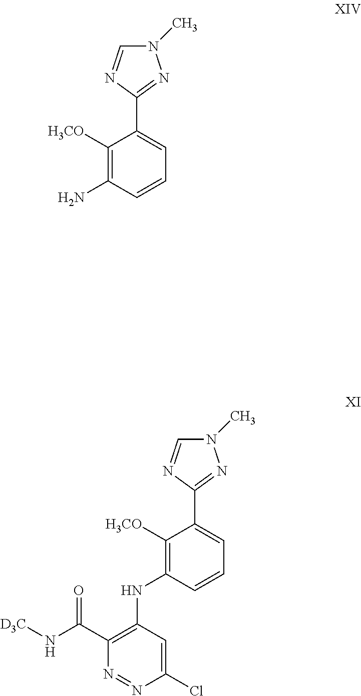 [SMILES: COc1c(N)cccc1-c1ncn(C)n1 ; [2H]C([2H])([2H])NC(=O)c1nnc(Cl)cc1Nc1cccc(-c2ncn(C)n2)c1OC]

(c-3) reacting the compound XI with 1-cyclopropanecarboxamide in the presence of a reagent and a base; wherein the reagent is selected from a precatalyst, or a catalyst and a ligand; and the base is selected from sodium carbonate or potassium carbonate, to obtain deucravacitinib, the compound I, in a purity of ≥95%, and wherein the content of impurity A represented by the following chemical structure,

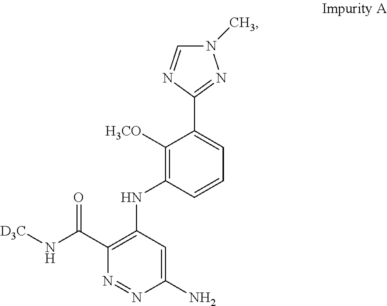 [SMILES: [2H]C([2H])([2H])NC(=O)c1nnc(N)cc1Nc1cccc(-c2ncn(C)n2)c1OC]

is less than 0.15% w/w as determined by High Performance Liquid Chromatography (HPLC).

In one embodiment, in the step (a-3), the salt of deuterated methylamine is deuterated methylamine hydrochloride.

In one embodiment, the coupling agent used in the step (a-3) is as described above for the step (a).

In one embodiment, in the step (a-3), the compound V, wherein B is Li, is reacted with deuterated methylamine or a salt thereof.

In one embodiment, the step (a-3) is carried out in the presence of a base.

In one embodiment, the base is selected from an inorganic base or an organic base or a mixture thereof.

In one embodiment, the inorganic base is selected from the group consisting of potassium tert-butoxide, sodium tert-butoxide, lithium tert-butoxide, lithium carbonate, sodium carbonate, potassium carbonate, sodium bicarbonate, potassium bicarbonate, cesium carbonate, tripotassium phosphate, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), and N,N,N′,N′-tetramethyl-1,8-naphthalenediamine, TMG (1,1,3,3-tetramethylguanidine), and a mixture thereof.

In one embodiment, the base is selected from the group consisting of diisopropylethylamine, triethylamine, tributylamine, triphenylamine, pyridine, lutidine, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), and a mixture thereof.

In one embodiment, the base used in the step (a-3) is diisopropylethylamine.

In one embodiment, the step (a-3) is carried out in the presence of a solvent.

In one embodiment, the solvent includes, but is not limited to, haloalkane solvents such as dichloromethane, chloroform, ethylene dichloride, and the like; ether solvents such as diethyl ether, diisopropyl ether, methyl tert-butyl ether, tetrahydrofuran, dioxane and the like; ester solvents such as methyl acetate, ethyl acetate, n-propyl acetate, isopropyl acetate, tert-butyl acetate and the like; hydrocarbon solvents such as toluene, xylene, chlorobenzene, heptane, hexane and the like; ketone solvents such as acetone, ethyl methyl ketone, methyl isobutyl ketone and the like; alcohol solvents such as methanol, ethanol, n-propyl alcohol, isopropyl alcohol, n-butyl alcohol, isobutyl alcohol, sec-butyl alcohol, tert-butyl alcohol, pentanol, octanol and the like; nitrile solvents such as acetonitrile, propionitrile, butyronitrile, and the like; dimethyl sulfoxide; dimethylacetamide; N-methyl-2-pyrrolidone; water; or mixtures thereof.

In one embodiment, the solvent is selected form a haloalkane solvent, an ester solvent, an ether solvent or nitrile solvent.

In one embodiment, the solvent used in the step (a-3) is a haloalkane solvent.

In one embodiment, the step (a-3) of the process for the preparation of deucravacitinib is carried out at a temperature of about −5° C. to about 0° C.

In one embodiment, the step (a-3) is carried out over a period ranging from about 2 hours to about 10 hours, or longer.

In one embodiment, in the step (a-3), after completion of the reaction, an additional base in water is added to the reaction mixture to adjust the pH of the reaction mixture to about 7 to about 10, thereby generating a biphasic reaction mixture.

In one embodiment, the additional base is selected from the group consisting of sodium hydroxide, potassium hydroxide, sodium carbonate, potassium carbonate, sodium bicarbonate, and potassium bicarbonate.

In one embodiment, in the step (a-3), the biphasic reaction mixture is subjected to layer separation to obtain an organic layer and an aqueous layer, wherein the organic layer contains the compound XIII.

In one embodiment, in the step (a-3), the compound XIII contained in the organic layer is not isolated and carried forward as such for further reaction in the step (b-3).

In the context of the present invention, the term “not isolated” as used herein means that the compound referred to is not separated as a solid, and that the compound remains in the solution.

In one embodiment, the compound XIII obtained in step (a-3) is in-situ, and is carried forward to step (b-3).

In the context of the present invention, the term “in-situ” means the intermediate compound formed in the step referred to is not isolated.

In one embodiment, in the step (a-3), the compound XIII contained in the organic layer is isolated by removal of the solvent.

In one embodiment, in the step (a-3), the removal of solvent may be accomplished by substantially complete evaporation of the solvent; or concentrating the solution, or cooling the solution, if required and then filtering the resulting solid.

In an embodiment of the present invention, the compound XIII prepared by the process as described herein is obtained in a purity of ≥99.0% as determined by HPLC.

The process described in the step (a-3) involves reaction of the compound V and the deuterated methylamine or a salt thereof (collectively referred to as “starting materials”) at a temperature of about −10° C. to about 0° C. The temperature of about −10° C. to about 0° C. is maintained throughout the reaction process, which includes maintaining the temperature of the reaction mixture during addition of the starting materials, during addition of the coupling agent, further, during addition of the base, and thereafter during stirring of the reaction mixture leading to the formation of the compound XIII. The mode of addition of the starting materials, the catalyst and the base may be varied while maintaining the temperature of about −10° C. to about 0° C. The aforementioned temperature during the addition of the starting materials, the catalyst and the base ensures preventing degradation of the starting materials during the course of the reaction thereby leading to the formation of the compound XIII with high purity of ≥99.0% as determined by HPLC.

In one embodiment, the base used in the step (b-3) is selected from an inorganic base or an organic base.

In one embodiment, the inorganic base is selected from the group consisting of potassium tert-butoxide, sodium tert-butoxide, lithium tert butoxide, lithium carbonate, sodium carbonate, potassium carbonate, sodium bicarbonate, potassium bicarbonate, cesium carbonate, tripotassium phosphate, sodium methoxide, potassium methoxide, sodium ethoxide, potassium ethoxide, LiHMDS (lithium bis(trimethylsilyl)amide), KHMDS (potassium bis(trimethylsilyl)amide), NaHMDS (sodium bis(trimethylsilyl)amide), lithium hexamethyldisilyazide, and a mixture thereof.

In one embodiment, the organic base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), N,N,N′,N′-tetramethyl-1,8-naphthalenediamine, TMG (1,1,3,3-tetramethylguanidine), and a mixture thereof.

In one embodiment, the base used in the step (b-3) is an organic base.

In one embodiment, the base used in the step (b-3) is an organic base, which is selected from diisopropylethylamine, or triethylamine.

In one embodiment, the reaction of the compound XIII with the compound XIV is carried out in the presence of a solvent.

In one embodiment, the solvent includes, but is not limited to, alcohol solvents such as methanol, ethanol, n-propyl alcohol, isopropyl alcohol, n-butyl alcohol, isobutyl alcohol, sec-butyl alcohol, tert-butyl alcohol, pentanol, octanol and the like; haloalkane solvents such as dichloromethane, chloroform, ethylene dichloride, and the like; ether solvents such as diethyl ether, diisopropyl ether, methyl tert-butyl ether, tetrahydrofuran, dioxane and the like; ester solvents such as methyl acetate, ethyl acetate, n-propyl acetate, isopropyl acetate, tert-butyl acetate and the like; hydrocarbon solvents such as toluene, xylene, chlorobenzene, heptane, hexane and the like; ketone solvents such as acetone, ethyl methyl ketone, methyl isobutyl ketone and the like; nitrile solvents such as acetonitrile, propionitrile, butyronitrile, and the like; dimethyl sulfoxide; dimethylacetamide; N-methyl-2-pyrrolidone; water; or mixtures thereof.

In one embodiment, the solvent used is an alcohol solvent.

In one embodiment, the alcohol solvent is selected from methanol, ethanol, n-propyl alcohol, isopropyl alcohol, n-butyl alcohol, isobutyl alcohol, sec-butyl alcohol, tert-butyl alcohol, pentanol, or octanol.

In one embodiment, the step (b-3) is carried out at a temperature of about 50° C. to about 120° C.

In one embodiment, the step (b-3) is carried out over a period ranging from about 3 hours to about 24 hours, or longer.

In one embodiment, the catalyst used in the step (c-3), is as described above for the step (b).

In one embodiment, in the step (c-3), the catalyst used is Pd₂(dba)₃.

In one embodiment, in the step (c-3), the ligand used is phosphine ligand, which is as described above for the step (2).

In one embodiment, in the step (c-3), the precatalyst used is as described above for the step (2).

In one embodiment, the reaction mixture of step (c-3) is purged with nitrogen or argon.

In one embodiment, the step (c-3) is carried out at a temperature of about 50° C. to about 130° C.

In one embodiment, the step (c-3) is carried out over a period ranging from about 3 hours to about 16 hours, or longer.

In one embodiment, the step (c-3) is carried out in the presence of a solvent.

In one embodiment, the solvent includes but is not limited to hydrocarbon solvents such as toluene, xylene, chlorobenzene, heptane, hexane and the like; nitrile solvents such as acetonitrile, propionitrile, butyronitrile, and the like; ether solvents such as dioxane, tetrahydrofuran, and the like or mixtures thereof.

In an embodiment of the present invention, the deucravacitinib prepared by the process as described herein is obtained in a purity of ≥95% as determined by HPLC, and wherein the content of Impurity A as described above, is less than 0.15% w/w relative to the amount of deucravacitinib, the compound I, as determined by HPLC.

The process described in the step (c-3) involves reaction of the compound XI with 1-cyclopropanecarboxamide in the presence of a base, wherein the base is selected from sodium carbonate or potassium carbonate. It was found that the use of sodium carbonate or potassium carbonate as the base provided advantage compared to caesium carbonate, the use of which is exemplified in the WO '661 publication. The use of sodium carbonate or potassium carbonate ensured that the content of Impurity A is less than 0.15% w/w relative to the amount of deucravacitinib, the compound I, as determined by HPLC.

Whereas, when caesium carbonate is used as a base in the reaction of the compound XI with 1-cyclopropanecarboxamide, it results in formation of the Impurity A in a higher amount thereby affect the overall purity of the desired compound, deucravacitinib.

In one embodiment, the present invention provides deucravacitinib wherein the content of Impurity A as described above, is less than 0.15% w/w relative to the amount of deucravacitinib, obtained by above process, as analyzed for chemical purity using high performance liquid chromatography (HPLC) with the conditions described below:
Reagents and Solvents: Ammonium Acetate (AR grade), Acetonitrile (Gradient grade), Methanol (HPLC grade), Water (Milli Q or equivalent), Glacial Acetic Acid (AR grade)

### Chromatographic Conditions:

Apparatus: A High Performance Liquid Chromatograph equipped with quaternary gradient pumps, variable wavelength UV detector attached with data recorder and integrator software.
Column: X Bridge C18, 150×4.6 mm, 3.5 μm; Welch Ghostbuster Column: 50×4.6 mm to be attached between solvent mixing chamber and sample injector.
Column Oven Temperature: 30° C.
Sample Cooler Temperature: 10° C.

### Mobile Phase Preparation:

Buffer: 0.01M Ammonium Acetate in water, pH adjust to 6.60 with diluted Ammonia solution or Acetic Acid Solution.
Mobile Phase A: Buffer:Methanol (90:10) v/v
Mobile phase B: Acetonitrile (100%)

### Gradient Program:

| Time (min.) | % Mobile Phase A | % Mobile Phase B |
|---|---|---|
| 0.01 | 92 | 08 |
| 02 | 92 | 08 |
| 30 | 75 | 25 |
| 40 | 45 | 55 |
| 49 | 45 | 55 |
| 50 | 92 | 08 |
| 55 | 92 | 08 |

Diluent: Water:Acetonitrile:Methanol (40:30:30, v/v/v)
Flow Rate: 1.0 mL/minute
Detection: UV 240 nm
Injection Volume: 5.0 L
Run Time: 55.0 minutes
Needle Wash: Water:Acetonitrile:Methanol (20:40:40) v/v/v
Seal Wash: Water:Methanol (90:10, v/v)

The retention time of deucravacitinib is about 30.0 minutes under these conditions. Relative retention time for Impurity A is about 0.60 with respect to deucravacitinib.

In one embodiment, the present invention provides a process wherein deucravacitinib is obtained in a purity of ≥95%, and wherein the content of impurities designated herein as Impurity B, Impurity C, Impurity D and Impurity E represented by the following chemical structures;

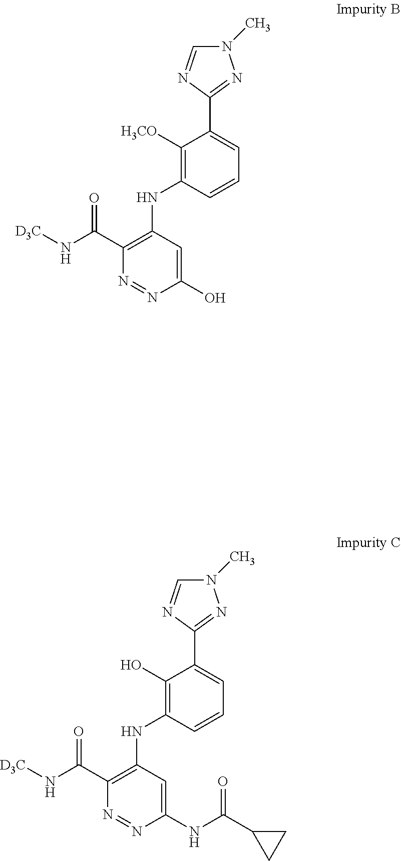 [SMILES: [2H]C([2H])([2H])NC(=O)c1nnc(O)cc1Nc1cccc(-c2ncn(C)n2)c1OC ; [2H]C([2H])([2H])NC(=O)c1nnc(NC(=O)C2CC2)cc1Nc1cccc(-c2ncn(C)n2)c1O]

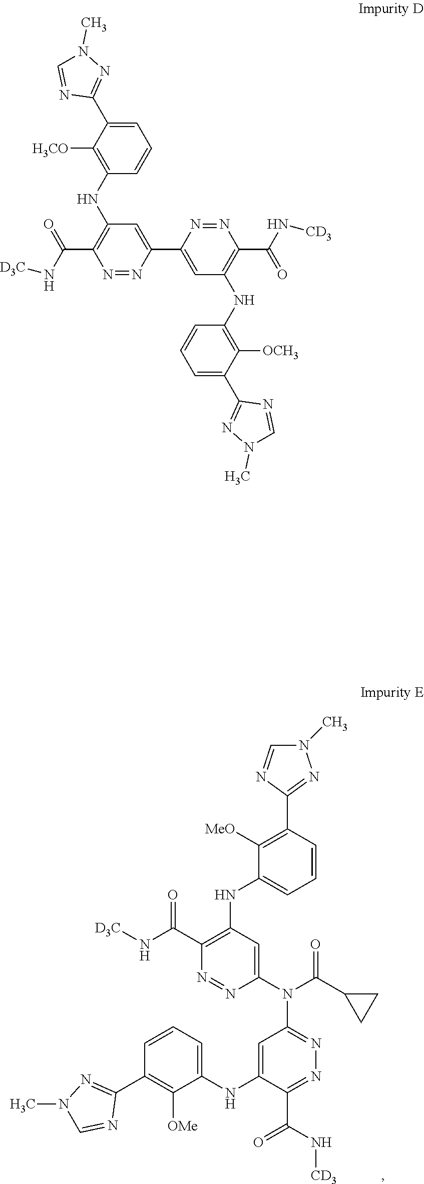 [SMILES: [2H]C([2H])([2H])NC(=O)c1nnc(-c2cc(Nc3cccc(-c4ncn(C)n4)c3OC)c(C(=O)NC([2H])([2H])[2H])nn2)cc1Nc1cccc(-c2ncn(C)n2)c1OC ; [2H]C([2H])([2H])NC(=O)c1nnc(N(C(=O)C2CC2)c2cc(Nc3cccc(-c4ncn(C)n4)c3OC)c(C(=O)NC([2H])([2H])[2H])nn2)cc1Nc1cccc(-c2ncn(C)n2)c1OC]

is less than 0.15% w/w relative to the amount of deucravacitinib, the compound I, as determined by HPLC.

In another aspect, the present invention provides an improved process for the preparation of crystalline form A of deucravacitinib (hereinafter referred to as the “crystalline form A”).

Crystalline form A of deucravacitinib is described in the WO '656 publication having X-ray powder diffraction pattern containing peaks at 7.8, 8.7, 10.1, 12.0, 12.4, 13.0, 15.8, 18.9, 19.3, and 20.4±0.2 degrees 2 theta.

In an embodiment, the process for the preparation of crystalline form A of deucravacitinib comprises:
(A) dissolving deucravacitinib in a first solvent selected from acetic acid or dimethyl sulfoxide, optionally in the presence of an additional solvent, at a temperature of about 40° C. to about 70° C. to form a solution;
(B) combining the solution of the step (A) with a second solvent selected from a lower alcohol, a nitrile solvent, water, or a mixture thereof, at a temperature of about 40° C. to about 70° C. to obtain a mixture;
(C) cooling the mixture of the step (B) to a temperature from about 30° C. to about 5° C.;
(D) obtaining crystalline form A of deucravacitinib from the mixture of the step (C); and
(E) isolating the crystalline form A of deucravacitinib as obtained in the step (D), wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta.

In the context of the present invention relating to the process for the preparation of the crystalline form A of deucravacitinib, the term “optionally” means dissolving deucravacitinib in a first solvent selected from acetic acid or dimethyl sulfoxide, either in the presence of an additional solvent or in the absence of an additional solvent.

In one embodiment, in the step (A), deucravacitinib is dissolved in a first solvent selected from acetic acid or dimethyl sulfoxide in the presence of an additional solvent at a temperature of about 40° C. to about 70° C. to form a solution.

In one embodiment, in the step (A), the additional solvent is selected from the group consisting of halogenated hydrocarbon solvent, ester solvent, and a mixture thereof.

In one embodiment, in the step (A), the additional solvent is selected from the group consisting of halogenated hydrocarbon solvent such as dichloromethane, dichloroethane, chloroform and the like; ester solvent such as methyl acetate, ethyl acetate, n-propyl acetate, isopropyl acetate, tert-butyl acetate and the like; and a mixture thereof.

In one embodiment, the additional solvent used in the step (A) is halogenated hydrocarbon solvent.

In one embodiment, the additional solvent used in the step (A) is halogenated hydrocarbon solvent. Preferably, the halogenated hydrocarbon solvent is dichloromethane.

In one embodiment, in the step (A), deucravacitinib is dissolved in a first solvent selected from acetic acid or dimethyl sulfoxide in the absence of an additional solvent at a temperature of about 40° C. to about 70° C. to form a solution.

In one embodiment, the step (A) of the process for the preparation of crystalline form A is carried out at a temperature of about 50° C. to about 60° C.

In one embodiment, the step (A) of the process for the preparation of crystalline form A is carried out by stirring the solution of deucravacitinib in the first solvent, optionally in the presence of the additional solvent, for any desired time period to achieve a complete dissolution of deucravacitinib. The stirring time may range from about 30 minutes to about 10 hours, or longer. The solution may be optionally filtered to get a particle-free solution.

In the context of the present invention relating to the process for the preparation of the crystalline form A of deucravacitinib, the term “combining” means adding the solution of step (A) to the second solvent selected from a lower alcohol, a nitrile solvent, water, or a mixture thereof; or adding the second solvent selected from the lower alcohol, the nitrile solvent, water, or mixture thereof to the solution of step (A).

As used herein, the term “lower alcohol” means C₁-C₄ alkyl alcohol, wherein alkyl includes linear and branched alkyl.

In one embodiment, the lower alcohol is selected from the group consisting of methyl alcohol, ethyl alcohol, n-propyl alcohol, isopropyl alcohol, n-butyl alcohol, isobutyl alcohol, sec-butyl alcohol, and tert-butyl alcohol.

In one embodiment, the nitrile solvent is selected from the group consisting of acetonitrile, propionitrile, and butyronitrile.

In one embodiment, the step (B) of the process for the preparation of crystalline form A is carried out at a temperature of about 50° C. to about 60° C.

In one embodiment, when the first solvent used in the step (A) is acetic acid, then the second solvent used in the step (B) is water.

In one embodiment, in the process for the preparation of crystalline form A, the first solvent used in the step (A) is acetic acid, the additional solvent used in the step (A) is dichloromethane, and the second solvent used in the step (B) is water.

In one embodiment, the step (C) of the process for the preparation of crystalline form A is carried out at a temperature of about 30° C. to about 20° C.

In one embodiment, the step (C) of the process for the preparation of crystalline form A is carried out at a temperature of about 15° C. to about 10° C.

In one embodiment, the step (C) of the process for the preparation of crystalline form A is carried out by stirring the mixture of step (B) for a suitable time. The stirring time may range from about 30 minutes to about 5 hours, or longer.

In one embodiment, the step (D) of the process for the preparation of the crystalline form A involving obtaining the crystalline form A comprises stirring the mixture of step (C).

In one embodiment, in the step (D), the mixture of the step (C) may be stirred for a suitable time. The stirring time may range from about 30 minutes to about 5 hours, or longer.

In one embodiment, in the step (E) of the process for the preparation of the crystalline form A, the resulting crystalline form A of deucravacitinib is isolated by any method known in the art. The method, may involve any of techniques, known in the art, including filtration by gravity or by suction, centrifugation, and the like.

In one embodiment, the isolated crystalline form A of deucravacitinib may be further dried. Drying may be suitably carried out in an equipment conventionally used in the art for the purpose, such as a tray drier, a vacuum oven, an air oven, a fluidized bed drier, a spin flash drier, a flash drier and the like. The drying may be carried out at a temperature ranging from about room temperature to about 100° C. with or without vacuum. The drying may be carried out for any desired time until the required product quality is achieved. The drying time may vary from about 1 hour to about 25 hours, or longer.

In another embodiment, the process for the preparation of crystalline form A of deucravacitinib comprises:
(A-i) dissolving deucravacitinib in a first solvent selected from acetic acid or dimethyl sulfoxide, optionally in the presence of an additional solvent, at a temperature of about 20° C. to about 35° C. to form a solution;
(B-i) combining the solution of step (A-i) with a second solvent selected from a lower alcohol or water, at a temperature of about 20° C. to about 35° C. to obtain a mixture;
(C-i) obtaining crystalline form A of deucravacitinib from the mixture of step (B-i); and
(D-i) isolating the crystalline form A of deucravacitinib as obtained in the step (C-i),
wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta; and
wherein when the first solvent is acetic acid, then the second solvent is not water.

In one embodiment, when the first solvent used in the step (A-i) is acetic acid, then the second solvent used in the step (B-i) is a lower alcohol, wherein the lower alcohol is methanol.

In one embodiment, when the first solvent used in the step (A-i) is acetic acid, then the second solvent used in the step (B-i) is a lower alcohol, wherein the lower alcohol is isopropyl alcohol.

In one embodiment, in the process for the preparation of crystalline form A, the first solvent used in the step (A-i) is acetic acid, the additional solvent used in the step (A-i) is dichloromethane, and the second solvent used in the step (B-i) is a lower alcohol, wherein the lower alcohol is methanol.

In one embodiment, in the process for the preparation of crystalline form A, the first solvent used in the step (A-i) is acetic acid, the additional solvent used in the step (A-i) is dichloromethane, and the second solvent used in the step (B-i) is a lower alcohol, wherein the lower alcohol is isopropyl alcohol.

In one embodiment, when the first solvent used in the step (A-i) is dimethyl sulfoxide, then the second solvent used in the step (B-i) is water.

In another embodiment, the process for the preparation of crystalline form A of deucravacitinib comprises:
(A-iii) dissolving deucravacitinib in dimethylsulfoxide to form a solution;
(B-iii) combining the solution of the step (A-iii) with a lower alcohol, a nitrile solvent, water, or a mixture thereof to obtain a mixture;
(C-iii) obtaining crystalline form A from the mixture of the step (B-iii); and
(D-iii) isolating the crystalline form A as obtained in the step (C-iii), wherein the crystalline form A is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta.

In one embodiment, the step (A-iii) of the process for the preparation of the crystalline form A is carried out at a temperature ranging from about 25° C. to about 70° C.

In one embodiment, the step (B-iii) of the process for the preparation of the crystalline form A is carried out at a temperature ranging from about 25° C. to about 35° C.

In one embodiment, the step (C-iii) of the process for the preparation of the crystalline form A is carried out at a temperature ranging from about 25° C. to about 35° C.

The present invention also provides novel crystalline forms of deucravacitinib as described herein.

Crystalline forms of deucravacitinib namely the form GL1 and the form GL2 of the present invention can be characterized by different analytical parameters, alone or in combination such as, but not limited to, X-ray powder diffraction (XRPD) pattern peaks at 2-theta values; IR spectrum; Thermogravimetric Analysis (TGA) thermogram, and/or any other method of characterization of a solid state form such as solid state ¹³C NMR spectrum; and Raman spectrum that are known to a person skilled in the art.

Crystalline forms of deucravacitinib namely the form GL1 and the form GL2 of the present invention are characterized by X-ray powder diffraction (XRPD) method as described herein.

In the context of the present invention, the term “substantially illustrated” as used in reference to Figure(s) 1, 2 and 3 relating to the crystalline forms of deucravacitinib as described herein, mean that for comparison purposes some variability in peak intensities from those shown in the Figures is allowed. It will be understood by a person skilled in the art that the 2-theta values of the XRPD pattern may vary slightly from one instrument to another, and also depending on variations in sample preparation and batch to batch variation. Therefore, the 2 theta values are not to be construed as absolute. Person skilled in the art will also understand that the relative intensities of peaks may vary to certain extent depending on orientation effects.

In one aspect, the present invention provides a crystalline form GL1 of deucravacitinib, which is characterized by an X-ray powder diffraction (XRPD) pattern having peaks at 3.34, 7.71, 12.1, 23.4, and 25.6±0.2 degrees 2 theta.

In one embodiment as may be combined with the preceding paragraph, the present invention provides a crystalline form GL1 of deucravacitinib further characterized by an X-ray powder diffraction (XRPD) pattern having any one, two or three additional peaks selected from the group consisting of 6.7, 15.1, 26.7 and 27.5±0.2 degrees 2 theta.

In one aspect, the present invention provides a crystalline form GL1 of deucravacitinib, which is characterized by an X-ray powder diffraction (XRPD) pattern having peaks at 3.34, 6.7, 7.71, 12.1, 15.1, 23.4, 25.6, 26.7 and 27.5±0.2 degrees 2 theta.

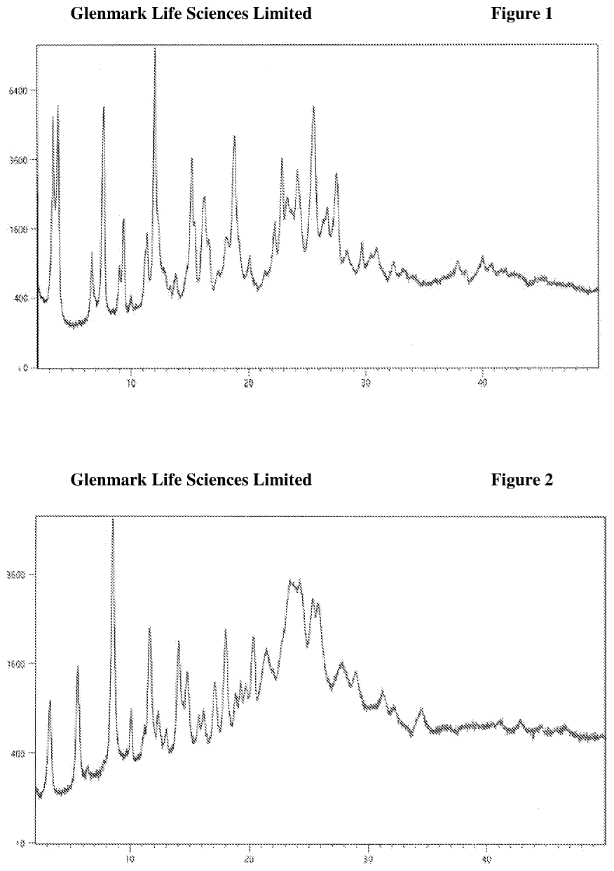

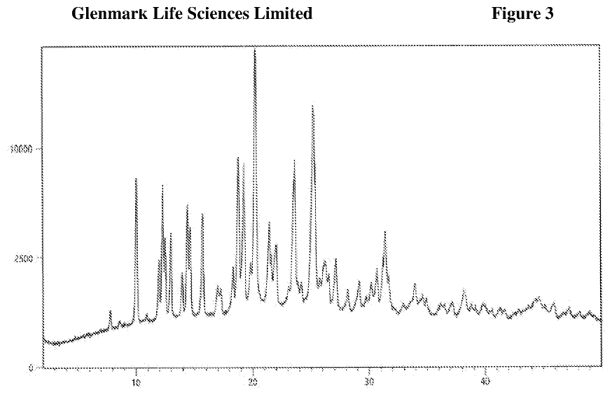

In one embodiment, the present invention provides a crystalline form GL1 of deucravacitinib characterized by an X-ray powder diffraction (XRPD) pattern as substantially illustrated in FIG. 1.

In one embodiment, the present invention provides a crystalline form GL1 of deucravacitinib, which is characterized by an X-ray powder diffraction (XRPD) pattern having peaks at 3.34, 7.71, 12.1, 23.4, and 25.6±0.2 degrees 2 theta, which is substantially in accordance with FIG. 1.

In one embodiment, the present invention provides a crystalline form GL1 of deucravacitinib wherein the water content is in the range of 3.7-7.0% by Karl Fisher method.

In one embodiment, the crystalline form GL1 of deucravacitinib obtained is a hydrate.

In another aspect, the present invention provides a process for the preparation of crystalline form GL1 of deucravacitinib, wherein the process comprises:
(a1) dissolving deucravacitinib in acetic acid to form a solution;
(b1) combining the solution of the step (a1) with water to obtain a mixture;
(c1) obtaining crystalline form GL1 from the mixture of the step (b1); and
(d1) isolating the crystalline form GL1 as obtained in the step (c1).

In one embodiment, the step (a1) of the process for the preparation of crystalline form GL1 is carried out at a temperature of about 25° C. to about 40° C.

In one embodiment, the step (a1) of the process for the preparation of crystalline form GL1 is carried out at a temperature of about 25° C. to about 35° C.

In one embodiment, the step (a1) of the process for the preparation of crystalline form GL1 is carried out by stirring the solution of deucravacitinib in acetic acid for any desired time period to achieve a complete dissolution of deucravacitinib. The stirring time may range from about 30 minutes to about 10 hours, or longer. The solution may be optionally filtered to get a particle-free solution.

In the context of the present invention relating to the process for the preparation of crystalline form GL1 of deucravacitinib, the term “combining” means adding the solution of the step (a1) to water, or adding water to the solution of the step (a1).

In one embodiment, the step (b1) of the process for the preparation of crystalline form GL1 is carried out at a temperature of about 25° C. to about 40° C.

In one embodiment, the step (b1) of the process for the preparation of crystalline form GL1 is carried out at a temperature of about 25° C. to about 35° C.

In one embodiment, the step (c1) of the process for the preparation of crystalline form GL1 involving obtaining crystalline form GL1 comprises stirring the mixture of step (b1).

In one embodiment, in the step (c1) of the process for the preparation of crystalline form GL1, the mixture of the step (b1) may be stirred for a suitable time. The stirring time may range from about 30 minutes to about 10 hours, or longer.

In one embodiment, in the process for the preparation of crystalline form GL1, stirring in the step (c1) may be continued for any desired time period to obtain the crystalline form GL1 of deucravacitinib.

In one embodiment, in the process for the preparation of crystalline form GL1, the step (c1) is carried out at a temperature of about 25° C. to about 35° C.

In one embodiment, in the step (d1) of the process for the preparation of crystalline form GL1, the resulting form GL1 of deucravacitinib is isolated by any method known in the art. The method, may involve any of techniques, known in the art, including filtration by gravity or by suction, centrifugation, and the like.

In one embodiment, the isolated crystalline form GL1 of deucravacitinib may be further dried. Drying may be suitably carried out in an equipment conventionally used in the art for the purpose, such as a tray drier, a vacuum oven, an air oven, a fluidized bed drier, a spin flash drier, a flash drier and the like. The drying may be carried out at a temperature ranging from about room temperature to about 60° C. with or without vacuum.

The drying may be carried out for any desired time until the required product quality is achieved. The drying time may vary from about 1 hour to about 25 hours, or longer.

In another aspect, the present invention provides a crystalline form GL2 of deucravacitinib characterized by an X-ray powder diffraction (XRPD) pattern having peaks at 8.5, 11.7, 18.0, 23.4, and 24.3±0.2 degrees 2 theta.

In one embodiment, as may be combined with the preceding paragraph, the present invention provides a crystalline form GL2 of deucravacitinib further characterized by an X-ray powder diffraction (XRPD) pattern having any one, two, three or four additional peaks selected from the group consisting of 3.3, 5.6, 17.1, and 27.7±0.2 degrees 2 theta.

In one aspect, the present invention provides a crystalline form GL2 of deucravacitinib characterized by an X-ray powder diffraction (XRPD) pattern having peaks at 3.3, 5.6, 8.5, 11.7, 17.1, 18.0, 23.4, 24.3, and 27.7±0.2 degrees 2 theta.

In one embodiment, the present invention provides a crystalline form GL2 of deucravacitinib characterized by an X-ray powder diffraction (XRPD) pattern as substantially illustrated in FIG. 2.

In one embodiment, the present invention provides a crystalline form GL2 of deucravacitinib characterized by an X-ray powder diffraction (XRPD) pattern having peaks at 8.5, 11.7, 18.0, 23.4, and 24.3±0.2 degrees 2 theta, which is substantially in accordance with FIG. 2.

In one embodiment, the present invention provides a crystalline form GL2 of deucravacitinib wherein the water content is in the range of 3.7-10.2% by Karl Fisher method.

In one embodiment, the crystalline form GL2 of deucravacitinib obtained is a hydrate.

In another aspect, the present invention provides a process for the preparation of crystalline form GL2 of deucravacitinib, wherein the process comprises:
(a2) dissolving deucravacitinib in acetic acid to form a solution;
(b2) combining the solution of the step (a2) with a lower alcohol and water to obtain a mixture;
(c2) obtaining crystalline form GL2 from the mixture of the step (b2); and
(d2) isolating the crystalline form GL2 as obtained in the step (c2).

In one embodiment, the step (a2) of the process for the preparation of crystalline form GL2, is carried out at a temperature of about 25° C. to about 40° C.

In one embodiment, the step (a2) of the process for the preparation of crystalline form GL2, is carried out at a temperature of about 25° C. to about 35° C.

In one embodiment, the step (a2) of the process for the preparation of crystalline form GL2, is carried out by stirring the solution of deucravacitinib in acetic acid for any desired time period to achieve a complete dissolution of deucravacitinib. The stirring time may range from about 30 minutes to about 10 hours, or longer. The solution may be optionally filtered to get a particle-free solution.

In the context of the present invention relating to the process for the preparation of crystalline form GL2 of deucravacitinib, the term “combining” means adding the solution of step (a2) to a lower alcohol and water; or adding a lower alcohol and water to the solution of step (a2).

In one embodiment, the lower alcohol is selected from the group consisting of methyl alcohol, ethyl alcohol, n-propyl alcohol, isopropyl alcohol, n-butyl alcohol, isobutyl alcohol, sec-butyl alcohol, and tert-butyl alcohol.

In one embodiment, the step (b2) of the process for the preparation of crystalline form GL2, is carried out at a temperature of about 25° C. to about 40° C.

In one embodiment, the step (b2) of the process for the preparation of crystalline form GL2, is carried out at a temperature of about 25° C. to about 35° C.

In one embodiment, the step (c2) of the process for the preparation of crystalline form GL2 involving obtaining crystalline form GL2 comprises stirring the mixture of step (b2).

In one embodiment, in the step (c2), the mixture of the step (b2) may be stirred for a suitable time. The stirring time may range from about 30 minutes to about 10 hours, or longer.

In one embodiment, stirring in the step (c2) may be continued for any desired time period to obtain the crystalline form GL2 of deucravacitinib.

In one embodiment, the step (c2) of the process for the preparation of crystalline form GL2 is carried out at a temperature of below about 25° C.

In the context of the present invention, the term “below about 25° C.” as used herein means the temperature ranging from about 25° C. to about 0° C., preferably about 25° C. to about 5° C.

In one embodiment, the step (c2) of the process for the preparation of crystalline form GL2 involving obtaining crystalline form GL2 comprises cooling and stirring the mixture of step (b2).

In one embodiment, the step (c2) of the process for the preparation of crystalline form GL2 is carried out by cooling the mixture of step (b2) from a temperature of about 25° C. to about 5° C.

In one embodiment, in the step (d2) of the process for the preparation of crystalline form GL2, the resulting form GL2 of deucravacitinib is isolated by any method known in the art. The method, may involve any of techniques, known in the art, including filtration by gravity or by suction, centrifugation, and the like.

In one embodiment, the isolated crystalline form GL2 of deucravacitinib may be further dried. Drying may be suitably carried out in an equipment conventionally used in the art for the purpose, such as a tray drier, a vacuum oven, an air oven, a fluidized bed drier, a spin flash drier, a flash drier and the like. The drying may be carried out at a temperature ranging from about room temperature to about 60° C. with or without vacuum. The drying may be carried out for any desired time until the required product quality is achieved. The drying time may vary from about 1 hour to about 25 hours, or longer.

In an embodiment, the present invention provides a process for the preparation of crystalline form A of deucravacitinib, wherein the process comprises:
(A-ii) providing a solution of a crystalline form of deucravacitinib in a first solvent selected from acetic acid or dimethyl sulfoxide, optionally in the presence of an additional solvent, at a temperature of about 40° C. to about 70° C.; wherein the crystalline form of deucravacitinib is selected from crystalline form GL-1 or crystalline form GL-2;
(B-ii) combining the solution of step (A-ii) with a second solvent selected from a lower alcohol, a nitrile solvent, water, or a mixture thereof, at a temperature of about 40° C. to about 70° C. to obtain a mixture;
(C-ii) cooling the mixture of step (B-ii) to a temperature from about 30° C. to about 5° C.;
(D-ii) obtaining crystalline form A of deucravacitinib from the mixture of step (C-ii); and
(E-ii) isolating the crystalline form A of deucravacitinib as obtained in the step (D-ii),
wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta.

In an embodiment of the present invention, the crystalline forms of deucravacitinib of the present invention prepared by the processes as described herein, are obtained in substantially pure form.

As used herein, the term “substantially pure,” when used in reference to the crystalline forms of deucravacitinib, indicates purity of the crystalline form greater than 90%, including greater than 90%, 91%, 92%, 93%, 94%, 95%, 96%, 97%, 98% and 99% as determined using high performance liquid chromatography (HPLC).

In one embodiment, the crystalline forms of deucravacitinib are obtained in a purity of ≥99%, and wherein the content of any of the impurities designated herein as Impurity A, Impurity B, Impurity C, Impurity D and Impurity E; as described above, is less than 0.15%.

In one embodiment, the crystalline forms of deucravacitinib are obtained in a purity of ≥99%, and wherein the content of any of the impurities, as described above, is less than 0.10%.

In one embodiment, the crystalline forms of deucravacitinib are obtained in a purity of ≥99%, and wherein the content of any of the impurities, as described above, is less than 0.05%.

In one embodiment, the crystalline forms of deucravacitinib are obtained in a purity of ≥99%, and wherein any of the impurities, as described above, is not detected.

In an embodiment, the crystalline forms of deucravacitinib of the present invention may have a D₉₀ particle size of less than about 250 microns, preferably less than about 150 microns, more preferably less than about 50 microns, still more preferably less than about 20 microns, still more preferably less than about 15 microns, and most preferably less than about 10 microns.

In an embodiment, the crystalline forms of deucravacitinib of the present invention may have a D₅₀ particle size of less than about 250 microns, preferably less than about 150 microns, more preferably less than about 50 microns, still more preferably less than about 20 microns, still more preferably less than about 15 microns, and most preferably less than about 10 microns.

The particle size disclosed herein can be obtained by, for example, any milling, grinding, micronizing or other particle size reduction method known in the art to bring the solid state deucravacitinib into any of the foregoing desired particle size range.

In an embodiment, the present invention relates to a pharmaceutical composition comprising a therapeutically effective amount of crystalline form of deucravacitinib selected from form GL1 or form GL2 and at least one pharmaceutically acceptable excipient.

In an embodiment, the present invention also relates to crystalline form of deucravacitinib selected from form GL1 or form GL2 for use in the treatment of immune-mediated diseases selected from moderate to severe plaque psoriasis, Crohn's disease, ulcerative colitis, lupus erythematosus, psoriatic arthritis or alopecia areata.

The present invention further relates to a pharmaceutical composition comprising a therapeutically effective amount of crystalline form A of deucravacitinib, wherein the crystalline form A of deucravacitinib is obtained by a process as described herein.

The examples that follow are provided to enable one skilled in the art to practice the invention and are merely illustrative of the invention. The examples should not be read as limiting the scope of the invention as defined in the features and advantages.

### EXAMPLES

### Example 1: Preparation of (6-chloro-4-((3-cyano-2-methoxyphenyl)amino) pyridazine-3-carbonyl)oxy) zinc (the Compound IV)

To the solution of water (20 mL) and isopropyl alcohol (5 mL) was added lithium 4,6-dichloropyridazine-3-carboxylate (4.05 g) and zinc acetate dihydrate (3.40 g) and 3-amino-2-methoxy benzontrile (the compound VI, 2.3 g) at about 25° C. The reaction mass was heated at about 65° C. for about 30 hr. After completion of reaction, water was added to the reaction mass. The resultant slurry mass was cooled to about 20° C., stirred for about 60 min and filtered. The wet cake obtained was washed with water and tetrahydrofuran, dried under vacuum at about 60° C. Yield: 4.26 g, (80%).

¹H NMR: (DMSO-d₆, 400 MHz): δ 12.13 (br s, 1H), 7.927 (d, 1H), 7.68 (d, 1H), 7.36 (t, 1H), 7.31 (s, 1H), 3.91 (s, 3H)

### Example 2: Preparation of 6-chloro-4-((3-cyano-2-methoxyphenyl)amino)-N-(trideuteriomethyl)pyridazine-3-carboxamide (the Compound III)

To (6-chloro-4-((3-cyano-2-methoxyphenyl)amino)pyridazine-3-carbonyl)oxy) zinc (2 g) was added acetonitrile (4 mL) and N-methyl pyrrolidinone (10 mL). The reaction mass was cooled to about 15° C. and added deuterated methylamine (0.50 g). The temperature of the reaction mass was raised to about 20° C. and stirred for 4 hr. To the reaction mass was added HOBt·H₂O (0.45 g) and EDC·HCl (0.28 g) at about 20° C. After completion of the reaction, water and methylene dichloride was added to the reaction mass. The mixture was stirred for about 20 min and layers were separated. Methylene dichloride layer was washed with 5% aqueous sodium bicarbonate solution and water. Methylene dichloride layer was distilled under vacuum at about 40° C. and the residue was degassed at about 55° C. The obtained crude mass was stirred in a mixture of isopropyl alcohol and water for about 3 hr at about 15° C. and filtered. The obtained wet solid was washed with water, dried under vacuum at about 65° C. Yield: 1.50 g (80%).

### Example 3: Preparation of 4-((3-cyano-2-methoxyphenyl) amino)-6-(cyclopropanecarboxamido)-N-(trideuteriomethyl)pyridazine-3-carboxamide (the Compound II)

To the reactor, was added acetonitrile (50 mL), toluene (70 mL), 6-chloro-4-((3-cyano-2-methoxyphenyl)amino)-N-(trideuteriomethyl)pyridazine-3-carboxamide (the compound III, 7 g), DBU (3.32 g), cyclopropane carboxamide (4.65 g), potassium carbonate (10.83 g) and Josiphos SL-J009-1 Pd G3 (0.63 g). the reaction mass was purged with nitrogen gas and heated to about 70° C. for about 12 hr. The reaction mass was cooled to about 25° C., added water and stirred for about 60 min at about same temperature. The slurry mass was filtered, obtained wet solid was washed with water. The obtained wet cake was dried on rotavapour under vacuum at about 60° C. Yield: 6.8 g (85%).

### Example 4: Preparation of 6-(cyclopropanecarboxamido)-4-((2-methoxy-3-(1-methyl-1H-1,2,4-triazol-3-yl)phenyl)amino)-N-(trideuteriomethyl)pyridazine-3-carboxamide (Deucravacitinib, the Compound I)

To a mixture of 4-((3-cyano-2-methoxyphenyl) amino)-6-(cyclopropanecarboxamido)-N-(trideuteriomethyl)pyridazine-3-carboxamide (the compound II, 0.40 g) and N-methyl-N-formylhydrazine (0.40 g) in tetrahydrofuran (10 mL) was added potassium tert-butoxide (0.58 g) at about 25° C. and the reaction mass was stirred for about 12 hr at about same temperature. Water and ethyl acetate was added to the reaction mass, stirred for about 20 min and the layers were separated. Ethyl acetate layer was distilled under vacuum at about 50° C. The crude mass was purified by column chromatography eluting with 5% methanol in ethyl acetate and obtained fractions were distilled under vacuum on rotavapour at about 50° C. to obtain pure deucravacitinib. Yield: 0.14 g (30%).

### Example 5: Preparation of 2-Methoxy-3-nitrobenzoic acid

To a suspension of 2-methoxy benzoic acid (5 g) and acetic anhydride (30 mL) was added nitric acid (15 mL) slowly for about 60 min by maintaining the temperature at about 0° C. to about 10° C. The resulting reaction mixture was stirred to about 5° C. to about 15° C. for about 60 min. After completion of the reaction, precipitated mass was filtered and washed with purified water. The wet cake (5.3 g) was recrystallized once with water and twice with ethyl acetate to obtain solid. The obtained solid was dried in vacuum oven at about 45° C. to about 55° C. Yield: 1.16 g (18%)

### Example 6: Preparation of 2-Methoxy-3-nitrobenzamide

To a mixture of methylene dichloride (70 mL) and 2-methoxy-3-nitrobenzoic acid (7 g) was added triethylamine (7.17 g). The reaction mixture was cooled to about 5° C. to about 10° C. and thionyl chloride (6.33 g) in methylene dichloride (14 mL) was added slowly to it. The reaction mass was stirred at about 5° C. to about 10° C. for about 60 min and ammonia gas was passed slowly into the reaction mass for about 180 min. After completion of reaction, water was added to the reaction mass at about 20° C. to 25° C. and stirred for about 20-25 min. The layers were separated, organic layer was washed with 5% aqueous sodium bicarbonate and distilled under vacuum at about 40° C. The obtained residue was degassed under vacuum at about 55° C. for about 60 min. Yield: 4.18 g (60%).

### Example 7: Preparation of 3-amino-2-methoxybenzamide

2-Methoxy-3-nitrobenzamide (4 g), methanol (100 mL) and 10% Pd/C (0.4 g) was added to an autoclave and reaction mass was flushed thrice with nitrogen (0.5 kg). The reaction mass was heated to about 70° C. and stirred for about 15 hr in the presence of hydrogen gas (5 kg) at about 70° C. After completion of the reaction, reaction mass was filtered through celite and the celite bed was washed with methanol. The filtrate was concentrated and the obtained residue was degassed. Yield: 3.1 g (92%).

### Example 8: Preparation of 3-amino-2-methoxybenzonitrile (the Compound VI)

To a 3-amino-2-methoxybenzamide (3 g) was added POCl₃ (12 mL) slowly by maintaining the temperature at about below 80° C. The reaction mass was heated at about 80° C. and maintained for about 12 hr at about the same temperature. After completion of the reaction, the reaction mass was distilled under vacuum at about 60° C. to about 70° C. and water was added to the concentrated reaction mass at about 35° C. The pH of the reaction mass was adjusted to 10 by using aqueous solution of sodium carbonate. The precipitated mass was stirred for about 60 min, filtered and washed with water. The obtained wet solid was dried under vacuum for about 15 hr. Yield: 1.87 g (70%).

### Example 9: Preparation of 5-chloro-2-methoxy-3-nitrobenzoic acid

To a solution of 5-chloro-2-methoxybenoic acid (5 g) in concentrated sulfuric acid (17.5 mL) was added nitric acid (2.01 g) slowly at about 0° C. to about 10° C. and reaction mixture was stirred for about 2 hr at about same temperature. After completion of the reaction, the reaction mass was quenched in chilled water by maintaining the temperature at about 0° C. to about 10° C. The precipitated mass was stirred at about 10° C. for about 60 min and filtered. The obtained wet cake washed with water and dried under vacuum at about 55° C. for about 12 hr. Yield: 5.83 g (94%).

### Example 10: Preparation of 5-chloro-2-methoxy-3-nitrobenzamide

To a mixture of methylene dichloride (55 mL) and 5-chloro-2-methoxy-3-nitrobenzoic acid (5.8 g) was added triethylamine (5.05 g). The reaction mixture was cooled to about 5° C. to about 10° C. and thionyl chloride (4.46 g) in methylene dichloride (2.9 mL) was slowly added to it. The reaction mass was stirred at about 20° C. for about 120 min and ammonia gas was passed slowly into the reaction mass for about 180 min. After completion of reaction, water was added to the reaction mass at about 20° C. to 25° C. and stirred for about 20-25 min. The layers were separated, organic layer was washed with 5% aqueous sodium bicarbonate and distilled under vacuum at about 40° C. The obtained solid was isolated in a mixture of isopropyl alcohol and water. The wet cake was degassed under vacuum at about 55° C. Yield: 3.46 g (60%).

### Example 11: Preparation of 3-amino-2-methoxybenzamide

5-chloro-2-methoxy-3-nitrobenzamide (3.4 g), methanol (28 mL), sodium hydroxide (0.59 g) and Raney Nickel (5.17 g) was added to an autoclave and reaction mass was flushed with nitrogen (0.5 kg). The reaction mass was heated to about 75° C. and stirred for about 15 hr in the presence of hydrogen gas (1 kg). After completion of the reaction, reaction mass was filtered through celite and the celite bed was washed with methanol. The filtrate was concentrated and the obtained residue was degassed. Yield: 3.1 g.

### Example 12: Preparation of 3-amino-2-methoxybenzonitrile (the Compound VI)

To a 3-amino-2-methoxybenzamide (3.1 g) was added POCl₃ (6.3 mL) slowly by maintaining the temperature at about below 80° C. The reaction mass was heated at about 80° C. and maintained for about 4 hr at about the same temperature. After completion of the reaction, the reaction mass was distilled under vacuum and water was added to the concentrated reaction mass at about 35° C. The pH of the reaction mass was adjusted to 10 by using aqueous solution of sodium carbonate. The precipitated mass was stirred for about 90 min, filtered and washed with water. The obtained wet solid was dried under vacuum for about 12 hr at about 55° C. Yield: 1.44 g (65%).

### Example 13: Preparation of 5-bromo-2-methoxy-3-nitrobenzoic acid

To a solution of 5-bromo-2-methoxybenoic acid (5 g) in concentrated sulfuric acid (17.5 mL) was added nitric acid (2.01 g) slowly at about 0° C. to about 10° C. and reaction mixture was stirred for about 2 hr at about same temperature. After completion of the reaction, the reaction mass was quenched in chilled water by maintaining the temperature at about 0° C. to about 10° C. The precipitated mass was stirred at about 10° C. for about 60 min and filtered. The obtained wet cake washed with water and dried under vacuum at about 55° C. for about 12 hr. Yield: 5.97 g (95%).

### Example 14: Preparation of 5-bromo-2-methoxy-3-nitrobenzamide

To a mixture of methylene dichloride (56 mL) and 5-bromo-2-methoxy-3-nitrobenzoic acid (5.9 g) was added triethylamine (4.31 g). The reaction mixture was cooled to about 5° C. to about 10° C. and thionyl chloride (3.8 g) in methylene dichloride (3 mL) was slowly added to it. The reaction mass was stirred at about 20° C. for about 150 min and ammonia gas was passed slowly into the reaction mass for about 210 min at about 10° C. After completion of reaction, water was added to the reaction mass at about 20° C. to 25° C. and stirred for about 25 min. The layers were separated, organic layer was washed with 5% aqueous sodium bicarbonate and distilled under vacuum at about 40° C. The obtained solid was isolated in a mixture of isopropyl alcohol and water and stirred at about 25° C. for about 120 min. The obtained mass was filtered, washed with water and the wet cake was degassed under vacuum at about 55° C. for about 12 hr. Yield: 3.61 g (61.5%).

### Example 15: Preparation of 3-amino-2-methoxybenzamide

5-bromo-2-methoxy-3-nitrobenzamide (3.5 g), methanol (28 mL), sodium hydroxide (0.5 g) and Raney Nickel (5.25 g) was added to an autoclave and reaction mass was flushed with nitrogen (0.5 kg). The reaction mass was heated to about 75° C. and stirred for about 15 hr in the presence of hydrogen gas (1 kg). After completion of the reaction, reaction mass was filtered through celite and the celite bed was washed with methanol. The filtrate was concentrated and the obtained residue was degassed. Yield: 2.7 g.

### Example 16: Preparation of ((6-chloro-4-((3-cyano-2-methoxyphenyl) amino)pyridazine-3-carbonyl)oxy)zinc (the Compound IV)

To a solution of water (7.5 mL) and isopropyl alcohol (2 mL) was added 4,6-dichloropyridazine-3-carboxylic acid (0.78 g) and zinc acetate dihydrate (0.74 g) and 3-amino-2-methoxybenzonitrile (the compound VI, 0.5 g) at about 25° C. The reaction mass was heated at about 65° C. for about 30 hr. After completion of reaction, water was added to the reaction mass. The resultant slurry mass was cooled to about 20° C., stirred for about 60 min and filtered. The obtained wet cake was washed with water and tetrahydrofuran, dried under vacuum at about 60° C. Yield: 0.93 g (80%).

### Example 17: Preparation of ((6-chloro-4-((3-cyano-2-methoxyphenyl) amino)pyridazine-3-carbonyl)oxy)zinc (the Compound IV)

To a suspension of water (7.5 mL) and 4,6-dichloropyridazine-3-carboxylic acid (0.78 g) was charged sodium hydroxide (0.16 g). The suspension was stirred at about 25° C. To a clear solution of sodium salt of 4,6-dichloropyridazine-3-carboxylic acid were added zinc acetate dihydrate (0.74 g) and 3-amino-2-methoxy benzontrile (the compound VI, 0.5 g) at about 25° C. The reaction mass was heated at about 65° C. for about 30 hr. After completion of reaction, water was added to the reaction mass. The resultant slurry mass was cooled to about 20° C., stirred for about 60 min and filtered. The obtained wet cake was washed with water and tetrahydrofuran, dried under vacuum at about 60° C. Yield: 1.05 g (90.6%).

### Example 18: Preparation of 4-((3-cyano-2-methoxyphenyl) amino)-6-(cyclopropanecarboxamido)-N-(trideuteriomethyl)pyridazine-3-carboxamide (the Compound II)

To a round bottom flask, acetonitrile (3 mL), toluene (5 mL), 6-chloro-4-((3-cyano-2-methoxyphenyl)amino)-N-(trideuteriomethyl)pyridazine-3-carboxamide (the compound III, 1 g), DBU (0.47 g), cyclopropane carboxamide (0.66 g) and potassium carbonate (1.5 g) was added. Pd(OAc)₂ (10.5 mg) and Josiphos SL-J009-1 Pd G3 (90.8 mg) was added in acetonitrile (1 mL) and toluene (1 mL) in a smaple vial. and this mixture was purged with nitrogen gas for about 10 min and then added to the mixture of round bottom flask. The reaction mass was heated to about 70° C. for about 18 hr. After completion of the reaction, water and methylene dichloride was added and stirred for about 20 min. Methylene dichloride layer was separated and distilled under vacuum at about 40° C. and degassed. Crude solid was purified by column chromatography eluting with ethyl acetate and hexane (50:50). Yield: 0.69 g (60%)

### Example 19: Preparation of 6-chloro-4-((2-methoxy-3-(1-methyl-1H-1,2,4-triazol-3-yl)phenyl)amino)-N-(trideuteriomethyl)pyridazine-3-carboxamide (the Compound XI)

To a mixture of 6-chloro-4-((3-cyano-2-methoxyphenyl)amino)-N-(trideuteriomethyl)pyridazine-3-carboxamide (the compound III, 2.5 g) and N-methyl-N-formylhydrazine (4.32 g) in THF (50 mL) were added potassium tert-butoxide solution (55 mL, 1M in THF) at a temperature range of 0-10° C. The temperature of the reaction mass was raised to about 25° C. and the reaction mass was stirred for about 2 hr. The reaction mixture was concentrated under vacuum at a temperature of about 50° C. Water and methylene dichloride were added to the reaction mass and the mixture was stirred for about 20-30 min. The two layers were separated and the organic layer was concentrated under vacuum. The obtained solid was purified by column chromatography using silica gel 100-200 mesh and 75% ethyl acetate: 25% hexane. Yield: 1.17 g

### Example 20: Preparation of 6-(cyclopropanecarboxamido)-4-((2-methoxy-3-(1-methyl-1H-1,2,4-triazol-3-yl) phenyl) amino)-N-(trideuteriomethyl) pyridazine-3-carboxamide (Deucravacitinib, the Compound I)

To the reactor, was added acetonitrile (50 mL), toluene (70 mL), 6-chloro-4-((2-methoxy-3-(1-methyl-1H-1,2,4-triazol-3-yl)phenyl)amino)-N-(trideuteriomethyl) pyridazine-3-carboxamide (the compound XI, 0.75 g), cyclopropane carboxamide (0.42 g), DBU (0.30 g), potassium carbonate (0.98 g) and Josiphos SL-J009-1 Pd G3 (55 mg). The reaction mass was flushed with nitrogen gas and the temperature of the reaction mass was raised to about 75° C. and maintained for about 12 hr. The reaction mixture was concentrated under vacuum at a temperature of about 65° C. Water and methylene dichloride were added to the reaction mass and the mixture was stirred for about 20-25 min. The two layers were separated and the organic layer was concentrated under vacuum at a temperature of about 40° C. to about 50° C. The solid was purified by column chromatography using silica gel 100-200 mesh and 75% ethyl acetate: 25% hexane. Yield: 0.212 g

### Example 21: Preparation of 6-chloro-4-((2-methoxy-3-(1-methyl-1H-1,2,4-triazol-3-yl)phenyl)amino)-N-(methyl-d3)pyridazine-3-carboxamide (the Compound XI)

To a cooled solution of propylphosphonic acid (12.8 g) was added lithium 4,6-dichloropyridazine-3-carboxylate (log) at about 0° C. to about 5° C. and the reaction mixture was stirred for about 10 min. To the reaction mass was added deuterated methylamine hydrochloride (5.3 g) and stirred for about 10 min. To the above reaction mixture was added diisopropylethylamine (DIPEA, 16.2 g) at about 0° C. to about 5° C., stirred and maintained for about 10 hr. After completion of the reaction, methylene dichloride and 10% NaOH solution was added to the reaction mass till pH is adjusted to 7, stirred and both the layers are separated. Organic layer is washed with water and distilled under vacuum at a temperature of about below 40° C. to obtain solid mass. To the obtained mass, DMF (30 mL), 2-methoxy-3-(1-methyl-1H-1,2,4-triazol-3-yl)aniline (9.2 g), and DIPEA (8.1 g) was added. The reaction mixture was heated at about 95° C. to about 100° C. and maintained for about 5 hr. After completion of the reaction, reaction mixture was cooled to about 45° C. to about 50° C., added water and further cooled to about 25° C. to about 30° C. The reaction mixture was stirred for about 2 hr, filtered, wet solid was washed with purified water and dried in tray drier at about 55° C. Yield: 1.21 g (64.28%), HPLC Purity: 98%.

### Example 22: Preparation of Deucravacitinib

To a reactor, were added 6-chloro-4-((2-methoxy-3-(1-methyl-1H-1,2,4-triazol-3-yl) phenyl)amino)-N-(methyl-d3)pyridazine-3-carboxamide (log), cyclopropane carboxamide (5.63 g), Pd₂(dba)₃ (1.21 g), Xantphos (1.14 g), Na₂CO₃ (5.62 g), acetonitrile (40 mL) and toluene (80 mL). The reaction mass was flushed with nitrogen gas and the temperature of the reaction mass was raised to about 110° C. and maintained for about 16 hr. The reaction mixture was cooled to about 25° C. to about 30° C., added water, stirred for about 2 hr and filtered to obtain product. Yield: 9.2 g (81.63%), HPLC Purity: 95%.

### Example 23: Preparation of Crystalline Form GL1 of Deucravacitinib

To the deucravacitinib (1.93 g) was added acetic acid (10 mL), stirred at about 25° C. to obtain a solution and filtered through celite bed. To the solution was added purified water (30 mL) and stirred the obtained reaction mixture for about 18 hr at about 25° C. The reaction mass was filtered, washed with purified water and wet solid was dried at about 50° C. Yield: 1.3 g (67.35%), HPLC Purity: 99%.

### Example 24: Preparation of Crystalline Form GL1 of Deucravacitinib

To the deucravacitinib (3.2 g) was added acetic acid (12.6 mL), stirred at about 25° C. to obtain a solution and filtered through celite bed. To the solution was added purified water (38.4 mL) and stirred the obtained reaction mixture for about 23 hr at about 25° C. The reaction mass was filtered, washed with purified water and wet solid was dried at about 50° C. Yield: 1.95 g (61%), HPLC Purity: 98.75%.

### Example 25: Preparation of Crystalline Form GL1 of Deucravacitinib

To the deucravacitinib (1.5 g) was added acetic acid (6 mL), stirred at about 25° C. to obtain a solution and filtered through celite bed. To the solution was added purified water (18 mL) and stirred the obtained reaction mixture for about 4 hr at about 25° C. The reaction mass was filtered, washed with purified water and wet solid was dried at about 50° C. Yield: 0.87 g (58%), HPLC Purity: 99.7%.

### Example 26: Preparation of Crystalline Form GL1 of Deucravacitinib

To the deucravacitinib (1.6 g) was added acetic acid (6.4 mL), stirred at about 25° C. to obtain a solution and filtered through celite bed. To the solution was added purified water (19.2 mL) and stirred the obtained reaction mixture for about 3 hr at about 25° C. The reaction mass was filtered, washed with purified water and wet solid was dried at about 50° C. Yield: 0.88 g (55%), HPLC Purity: 99.24%.

### Example 27: Preparation of Crystalline Form GL2 of Deucravacitinib

To the deucravacitinib (1.93 g) was added acetic acid (10 mL), stirred at about 25° C. to obtain a solution and filtered through celite bed. To the solution was added isopropyl alcohol (5 mL) and purified water (20 mL). The obtained solution was cooled to about 5° C. and stirred for about 30 min. The temperature of the reaction mass was raised to about 20° C. and stirred for about 6 hr. The reaction mass was filtered, washed with purified water and wet solid was dried at about 45° C. for 16 hr. Yield: 1.1 g (57%), HPLC Purity: 99%.

### Example 28: Preparation of Crystalline Form A of Deucravacitinib

Deucravacitinib (1 g) was dissolved in dimethyl sulfoxide (5 mL) at about 65° C. to obtain a solution and filtered through celite bed. To the filtered solution was added isopropyl alcohol (10 mL) and then stirred the obtained reaction mixture for about 24 hr at about 25° C. The reaction mass was filtered, washed with purified water and wet solid was dried at about 45° C. Yield: 0.65 g (65%), HPLC Purity: 99.3%.

### Example 29: Preparation of Crystalline Form A of Deucravacitinib

To the deucravacitinib (7.9 g) was added dimethyl sulfoxide (15 mL) at about 25° C. and water (15 mL) was added. The solution was stirred for about 3 hr at about 25° C. The reaction mass was filtered, washed with purified water and wet solid was dried at about 50° C. to obtain product. Yield: 4.56 g (57%).

### Example 30: Preparation of Crystalline Form A of Deucravacitinib

Deucravacitinib (1.93 g) was dissolved in dimethyl sulfoxide (12 mL) at about 65° C. to obtain a solution and filtered through celite bed. To the filtered solution was added isopropyl alcohol (6 mL) and purified water (10 mL). The reaction mixture was cooled to about 25° C. and stirred for about 16 hr. The reaction mass was filtered, washed with isopropyl alcohol and wet solid was dried at about 45° C. Yield: 1.15 g (59.6%), HPLC Purity: 97.35%.

### Example 31: Preparation of Crystalline Form A of Deucravacitinib

Deucravacitinib (1.93 g) was dissolved in dimethyl sulfoxide (12 mL) at about 65° C. and added acetonitrile (6 mL) and water (12 mL). The reaction mixture was cooled to about 25° C. and stirred for about 4 hr. The reaction mass was filtered, washed with acetonitrile and water (1:1), and then wet solid was dried at about 45° C. Yield: 1.25 g (64.7%), HPLC Purity: 97.1%.

### Example 32: Preparation of 4,6-dichloro-N-(methyl-d3)pyridazine-3-carboxamide (the Compound XIII)

A mixture of propylphosphonic anhydride (T₃P in 50% ethyl acetate, 6.39 g) and methylene dichloride (10 mL) was cooled at about 0° C. to about −5° C. To this solution lithium 4,6-dichloropyridazine-3-carboxylate (1 g) was added and stirred for about 15 min. To this reaction mass, deuterated methylamine hydrochloride (0.42 g) was added at about 0° C. to about −5° C. and stirred for about 15 min. To this reaction mass, DIPEA (1.75 g) was added slowly at about 0° C. to about −5° C. The reaction mixture was stirred at about −5° C. to about 5° C. for about 1 hr to about 3 hr. After completion of the reaction, 5% aqueous NaOH solution was added and resulting biphasic mixture was stirred for about 30 min. Both the layers were separated and the aqueous layer was extracted with methylene dichloride. The organic layers were combined and washed with 5% aqueous NaOH solution and then with water, concentrated under vacuum to obtain light brown solid. Yield: 0.78 g (75%), HPLC Purity: 99.49%.

### Example 33: Preparation of 6-chloro-4-((2-methoxy-3-(1-methyl-1H-1,2,4-triazol-3-yl)phenyl)amino)-N-(methyl-d3)pyridazine-3-carboxamide (the Compound XI)

A mixture of 4,6-dichloro-N-(methyl-d3)pyridazine-3-carboxamide (the compound XIII, 1.5 g), 2-methoxy-3-(1-methyl-1H-1,2,4-triazol-3-yl)aniline (the compound XIV, 1.7 g), and DIPEA (1.1 g) in isopropyl alcohol (18 mL) was heated at about 80° C. to about 85° C. for about 24 hr. After completion of the reaction, reaction mixture was quenched with water (15 mL) and stirred for about 2 hr at about 25° C. The obtained slurry mass filtered, wet solid was washed with water and dried in air oven at about 50° C. to obtain white to off-white product. Yield: 2.16 g (80%), HPLC Purity: 99.5%.

### Example 34: Preparation of 6-chloro-4-((2-methoxy-3-(1-methyl-1H-1,2,4-triazol-3-yl)phenyl)amino)-N-(methyl-d3)pyridazine-3-carboxamide (the compound XI)

A mixture of propylphosphonic anhydride (T₃P in 50% ethyl acetate, 3.2 g) and methylene dichloride (5 mL) was cooled at about 0° C. to about −5° C. To this solution lithium 4,6-dichloropyridazine-3-carboxylate (0.5 g) was added and stirred for about 15 min. To this reaction mass, deuterated methylamine hydrochloride (0.21 g) was added at about 0° C. to about −5° C. and stirred for about 15 min. To this reaction mass, DIPEA (0.71 g) was added slowly at about 0° C. to about −5° C. The reaction mixture was stirred at about −5° C. to about 5° C. for about 1 hr to about 3 hr. After completion of the reaction, 10% aqueous NaOH solution was added and resulting biphasic mixture was stirred for about 30 min. Both the layers were separated and aqueous layer was extracted with methylene dichloride. Then combined organic layers were washed with water, concentrated under vacuum and striped out with isopropyl alcohol. To the resulting residue was added isopropyl alcohol (5 mL), 2-methoxy-3-(1-methyl-1H-1,2,4-triazol-3-yl)aniline (the compound XIV, 0.46 g) and DIPEA (0.4 g). The reaction mixture was heated at about 80° C. to about 85° C. for about 24 hr. After completion of the reaction, reaction mixture was quenched with water (6 mL) and stirred for about 2 hr at about 25° C. The obtained slurry mass filtered, wet solid was washed with water and dried in air oven at about 50° C. to obtain white to off-white product. Yield: 0.5 g (55%), HPLC Purity: 99.6%.

### Example 35: Preparation of Deucravacitinib

To a 4-neck round bottom flask, toluene (399 mL), 6-chloro-4-((2-methoxy-3-(1-methyl-1H-1,2,4-triazol-3-yl)phenyl)amino)-N-(methyl-d3)pyridazine-3-carboxamide (the compound XI, 21 g), cyclopropanecarboxamide (11.8 g), Na₂CO₃ (11.8 g), Xantphos (1.4 g) were added at about 25° C. and the resulting mixture was purged with nitrogen gas for about 15 min. To this reaction mixture, Pd₂(dba)₃ (1.5 g) was added. The reaction flask was flushed with toluene (21 mL) and purged with nitrogen gas for about 5 min. The reaction mixture was heated to about 110° C. and maintained at about same temperature for about 6 hr. After completion of the reaction, water (105 mL) was added and reaction mass was stirred for about 60 min. The reaction mass was filtered and the wet solid was washed with water and toluene. Yield: 18 g.

HPLC Purity: 97.44%

Related Substances by HPLC: Impurity A: Not detected

### Example 36: Preparation of Deucravacitinib

Deucravacitinib is prepared by following similar process as described in EXAMPLE 35 wherein Na₂CO₃ is replaced by K₂CO₃.

HPLC Purity: 96.70%

Related Substances by HPLC: Impurity A: Not Detected

### Comparative Example 1: Preparation of Deucravacitinib

Deucravacitinib is prepared by following similar process as described in EXAMPLE 35 wherein Na₂CO₃ is replaced by Cs₂CO₃.

HPLC Purity: 90.64%

Related Substances by HPLC: Impurity A: 0.81%

### Example 37: Preparation of Crystalline Form A of Deucravacitinib

The solid (5 g) was dissolved in methylene chloride (10 mL) and acetic acid (5 mL). To this solution methanol (60 mL) was added and the mixture was stirred at about 20° C. to about 25° C. for about 4 hr. The crystallized solid was filtered, washed twice with methanol, dried to obtain Form A of Deucravacitinib. Yield: 3.1 g (62%), HPLC Purity: 99.66%

### Example 38: Preparation of Crystalline Form A of Deucravacitinib

Deucravacitinib (5 g) was dissolved in acetic acid (12.5 mL) at about 50° C. to about 60° C. Activated carbon (0.5 g) was added to the clear solution at about 50° C. to about 60° C. and stirred at about 50° C. to about 60° C. for about 1 hr. The solution was filtered through hyflo Celite®, washed with acetic acid (2.5 mL). The filtrate was heated at about 50° C. to about 60° C. and water (75 mL) was added to the clear solution. The resulting mixture was stirred at about 50° C. to about 60° C. for about 30 min. The reaction mixture was cooled to about 15° C. to about 10° C. and maintained at about the same temperature for about 1 hr. The obtained product was filtered, washed twice with water and dried to obtain form A of deucravacitinib. Yield: 85%, HPLC Purity: 99.9%.

XRPD: 7.8, 8.7, 10.1, 12.0, 12.4, 13.0, 15.8, 18.9, 19.3, and 20.4±0.2 degrees 2 theta.

Particle Size: D₁₀: 9.4 μm, D₅₀: 21.64 μm, D₉₀: 39.49 μm; After jet milling: D₁₀: 1.6 μm, D₅₀: 3.0 μm, D₉₀: 5.6 μm

Stability data of crystalline form A of deucravacitinib prepared by the present invention:

| | | Storage Conditions | | |
|---|---|---|---|---|
| | | 25 °C/ | 40 °C/ | 60 °C/ |
| Test Parameter | Months | 60% RH | 75% RH | 90% RH |
| Polymorphic Form | 1 Month | Complies | Complies | Complies |
| Identification: | | with | with | with |
| The XRPD pattern | | Form A | Form A | Form A |
| should exhibit | | | | |
| characteristic | | | | |
| peaks of Form A at | | | | |
| 10.1°, 12.4°, 18.9°, | | | | |
| 19.3° and 20.4° ± | | | | |
| 0.2° 2 theta. | | | | |

1.-35. (canceled)

36. A process for the preparation of deucravacitinib, a compound of formula I (the “compound I”), 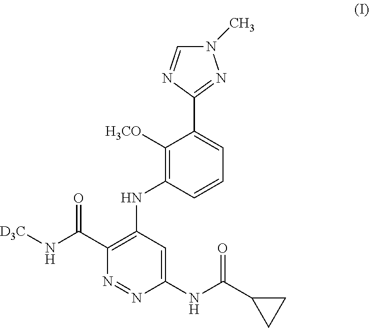 [SMILES: [2H]C([2H])([2H])NC(=O)c1nnc(NC(=O)C2CC2)cc1Nc1cccc(-c2ncn(C)n2)c1OC]
the process comprising the steps of:
(a-3) reacting a compound of formula V (the “compound V”), wherein B is selected from the group consisting of H, a C₁₋₆ alkyl, Li, Na, K, Mg, Ca and Zn; with deuterated methylamine of formula CD₃NH₂ or a salt thereof in the presence of a coupling agent at a temperature of about −10° C. to about 0° C. to obtain a compound of formula XIII (the “compound XIII”); 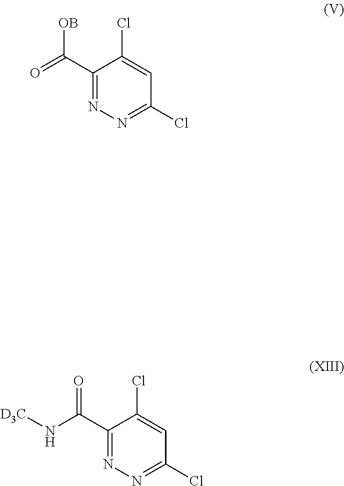
(b-3) reacting the compound XIII with a compound of formula XIV (the “compound XIV”) in the presence of a first base to obtain a compound of formula XI (the “compound XI”); and 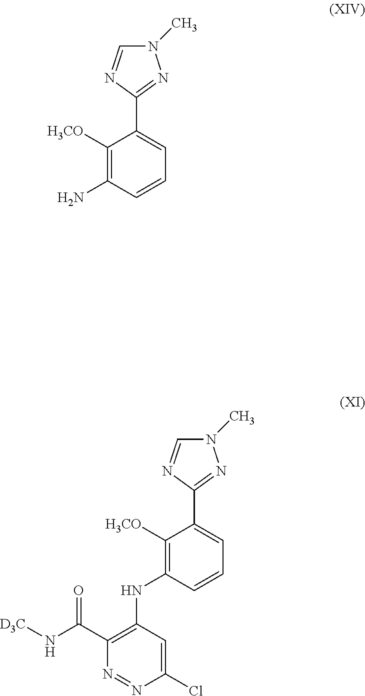 [SMILES: COc1c(N)cccc1-c1ncn(C)n1 ; [2H]C([2H])([2H])NC(=O)c1nnc(Cl)cc1Nc1cccc(-c2ncn(C)n2)c1OC]
(c-3) reacting the compound XI with 1-cyclopropanecarboxamide in the presence of a reagent and a second base; wherein the reagent is selected from a precatalyst, or a catalyst and a ligand; and the second base is selected from sodium carbonate or potassium carbonate, to obtain deucravacitinib, the compound I, in a purity of ≥95%, and having a content of an impurity A represented by the following chemical structure, 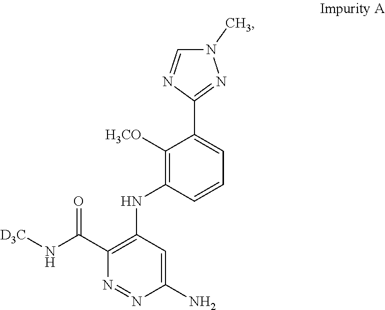 [SMILES: [2H]C([2H])([2H])NC(=O)c1nnc(N)cc1Nc1cccc(-c2ncn(C)n2)c1OC]
is less than 0.15% w/w as determined by High Performance Liquid Chromatography (HPLC).

37. The process of claim 36, wherein the coupling agent used in the step (a-3) is selected from the group consisting of T₃P (propylphosphonic anhydride), EDC (N-(3-dimethylaminopropyl)-N′-ethylcarbodiimide), DCC (dicyclohexylcarbodiimide), DIC (diisopropylcarbodiimide), HOBt (hydroxybenzotriazole), and a mixture thereof.

38. The process of claim 36, wherein the first base used in the step (b-3) is selected from the group consisting of diisopropylethylamine, triethylamine, tributylamine, triphenylamine, pyridine, lutidine, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), and a mixture thereof.

39. The process of claim 36, wherein the reagent used in the step (c-3) is a catalyst and a ligand, wherein the catalyst is a palladium catalyst selected from the group consisting of Pd(OAc)₂, PdCh(MeCN)₂, Pd₂(dba)₃, Pd(dba)₂, [(Allyl)PdCl]₂, [(Crotyl)PdCl]₂, and a mixture thereof, and the ligand is a phosphine ligand selected from Josiphos, SEGPHOS, SDP, Taniaphos, DPEphos, Xantphos, DPPF, DCyPF, or BINAP.

40. The process of claim 36, wherein the reagent used in the step (c-3) is a precatalyst, which is selected from Josiphos SL-J009-1 Pd G3, XPhos Pd G3, or XPhos Pd G2.

41. A process for the preparation of crystalline form A of deucravacitinib, wherein the process comprises:
(A) dissolving deucravacitinib in a first solvent selected from acetic acid or dimethyl sulfoxide, optionally in the presence of an additional solvent, at a temperature of about 40° C. to about 70° C. to form a solution;
(B) combining the solution of the step (A) with a second solvent selected from a lower alcohol, a nitrile solvent, water, or a mixture thereof, at a temperature of about 40° C. to about 70° C. to obtain a mixture;
(C) cooling the mixture of the step (B) to a temperature from about 30° C. to about 5° C.;
(D) obtaining crystalline form A of deucravacitinib from the mixture of the step (C); and
(E) isolating the crystalline form A of deucravacitinib as obtained in the step (D),
wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta.

42. The process of claim 41, wherein the step (A) is carried out in the presence of an additional solvent, which is selected from the group consisting of halogenated hydrocarbons, esters, and a mixture thereof.

43. A process for the preparation of crystalline form A of deucravacitinib, wherein the process comprises:
(A-i) dissolving deucravacitinib in a first solvent selected from acetic acid or dimethyl sulfoxide, optionally in the presence of an additional solvent, at a temperature of about 20° C. to about 35° C. to form a solution;
(B-i) combining the solution of the step (A-i) with a second solvent selected from a lower alcohol or water, at a temperature of about 20° C. to about 35° C. to obtain a mixture;
(C-i) obtaining crystalline form A of deucravacitinib from the mixture of the step (B-i); and
(D-i) isolating the crystalline form A of deucravacitinib as obtained in the step (C-i),
wherein the crystalline form A of deucravacitinib is characterized by an X-ray powder diffraction (XRPD) pattern having peaks at 7.8, 8.7, 10.1, 12.0, 12.4, 13.0, 15.8, 18.9, 19.3, and 20.4±0.2 degrees 2 theta; and
wherein when the first solvent is acetic acid, then the second solvent is not water.

44. The process of claim 43, wherein the step (A-i) is carried out in the presence of an additional solvent, which is selected from the group consisting of halogenated hydrocarbons, esters, and a mixture thereof.

45.-53. (canceled)

54. The process of claim 42, wherein the additional solvent is a halogenated hydrocarbon solvent selected from the group consisting of dichloromethane, dichloroethane, chloroform, and a mixture thereof.

55. The process of claim 42, wherein the additional solvent is an ester solvent selected from the group consisting of methyl acetate, ethyl acetate, n-propyl acetate, isopropyl acetate, tert-butyl acetate, and a mixture thereof.

56. The process of claim 41, wherein the second solvent used in the step (B) is a lower alcohol selected from the group consisting of methyl alcohol, ethyl alcohol, n-propyl alcohol, isopropyl alcohol, n-butyl alcohol, isobutyl alcohol, sec-butyl alcohol, tert-butyl alcohol and a mixture thereof.

57. The process of claim 41, wherein the second solvent used in the step (B) is a nitrile solvent selected from the group consisting of acetonitrile, propionitrile, butyronitrile, and a mixture thereof.
